(19) United States
(12) Patent Application Publication
(10) Pub. No.: US 2006/0142870 A1
(43) Pub. Date: Jun. 29, 2006

(54) Modular total ankle prosthesis apparatuses, systems and methods, and systems and methods for bone resection and prosthetic implantation

(76) Inventors: Shawn Robinson, Oceanside, CA (US); Mark Ray Foley, Escondido, CA (US); Robert J. Ball, San Marcos, CA (US); Michael Brage, Coto De Caza, CA (US); Lowell Harley Gill, Charlotte, NC (US); Christopher William DiGiovanni, Barrington, RI (US)

Correspondence Address: JENKINS, WILSON, TAYLOR & HUNT, P. A., 3100 TOWER BLVD, SUITE 1200, DURHAM, NC, 27707, US

(21) Appl. No.: 11/207,597

(22) Filed: Aug. 19, 2005

Related U.S. Application Data
(60) Provisional application No. 60/602,786, filed on Aug. 19, 2004.

(51) Int. Cl. A61F 2/42; A61B 17/17; A61B 17/90

(52) U.S. Cl. 623/21.18; 606/87; 606/96

(57) ABSTRACT

Ankle prosthesis apparatuses, systems and methods are provided as disclosed herein. Additionally, systems and methods for bone resection and implantation of prosthetics are provided, including surgical techniques and related instrumentation. An ankle prosthesis apparatus can include a talar component having a lower surface with a bone fixation portion for fixation to a talus bone and an upper surface designed for articulation with a bearing component. The bearing component can have a lower surface for articulation with the talar component and an upper surface for articulation with a tibial component. The tibial component can have a lower surface for articulation with the bearing component and an upper surface with a bone fixation portion for fixation to a tibia bone and/or a fibula bone. The bearing component can have a protrusion on its upper surface adapted for engagement with a recess on the tibial component to allow desired rotational and translational movement. Methods and systems can be used to prepare a bone surface for implantation of a prosthesis including determining a location for a curved cut line on the bone surface and drilling a series of holes tangent to the curved cut line to create a curved bone resection surface. Methods and systems can be used for the implantation of an ankle joint prosthesis including the use of an alignment guide, tibia and talus drill guides, tibia and talus saw guides, and tibia and talus broach guides, all components of which can be placed on and removed from a plurality of alignment anchor pins throughout the implantation procedure. A method for medially to laterally implanting an ankle joint prosthesis can include exposing tibia and talus bones from the medial side, resection of the tibia and talus bones, broaching the tibia and talus bones, and positioning and affixing the ankle joint prosthesis components.

### RELATED APPLICATION

This application claims the benefit of U.S. Provisional Patent Application Ser. No. 60/602,786, entitled Modular Total Ankle Prothesis Apparatuses and Methods, filed Aug. 19, 2004, the disclosure of which is incorporated herein by reference in its entirety.

### TECHNICAL FIELD

The present subject matter is directed generally to orthopedic prostheses. More specifically, the present subject matter is directed to ankle prosthesis apparatuses, systems and methods, and to systems and methods for bone resection and implantation of prosthesis apparatuses.

### BACKGROUND ART

The concept of total ankle arthroplasty has a long and relatively unsuccessful history. Only recently has total ankle arthroplasty regained some recognition as a viable treatment for limited indications. Replacement of an ankle joint can be particularly problematic due to the relatively small articular surfaces, complex biomechanics, limited access to the joint during replacement, and wide variation in patient candidacy. These factors have led to post-operative complications such as loosening, subsidence, pain, and prosthetic wear. In addition to these technical difficulties, regulatory agencies have classified ankle prosthetics in a manner substantially limiting scientific progress in ankle replacement due to the financial burden of obtaining market clearance for such devices.

Two types of ankle prosthetics that are generally available are semi-constrained and unconstrained prosthetics. Both types of prosthetics utilize a three-component design including an upper, middle, and lower component (tibial, bearing, and talar component, respectively).

A semiconstrained ankle prosthesis typically provides a tibial fixation component (usually metal), which provides firm attachment to the distal end of the tibia bone. A talar component provides firm attachment to the proximal end of the talus bone, and provides on its upper or proximal side a surface for articulation. A bearing component can fit between the tibial component and the talar component. The underside of the bearing can provide a surface to articulate with the surface of the talar component. These surfaces can be structured such that all motions present in a nature ankle can be at least partially replicated. Such motions can include plantar/dorsiflexion, rotation about the tibial axis, medial/lateral translation, and anterior/posterior translation. Rotations in the frontal region are usually not well supported as there is little curvature in this region. These motions can occur actively and lead to edge loading, causing higher stress and greater propensity for wear. Also, as the articular surfaces can be designed for mismatch, even under optimum implant positioning and loading, higher stress will be seen at the contact point due to the point loading associated with mismatched radii of the articular surfaces.

Unconstrained prosthetics are all generally the same in function. They are similar to semiconstrained prostheses except that the potential for motion between the tibial component and the bearing component is designed into the prosthesis. There is no intimate fit between the bearing component and the tibial component as the tibial component usually has a flat undersurface and the bearing component usually has a simple flat upper surface so that translation and rotation are allowed at this interface. Further, the interface between the talar component and the bearing component can have a curvature that is matched, so there is a large contact surface area and optimized contact stress that can result in reduced wear. This matched articulation can be accomplished because other motions are allowed for between the tibial and bearing components. It has been clearly shown with clinical history in all joints that if these motions are not allowed for, the force must be absorbed at the implant bone interface, and can lead to a greater propensity for loosening.

Current methods of bone surface preparation, such as resection of the tibia and talus bones for ankle joint prosthesis implantation, typically involve using a hand-held bone saw that is held by the surgeon for making the resection cut. These methods of bone resection have several disadvantages including over-cutting of the resection of the bone surfaces, initial misalignment of the cut, and performing cuts that are not straight throughout the length of the cut. These disadvantages can lead to longer healing time or more pain for the patient or performance problems of the prosthesis due to misalignment or improper contact between the implant components and the resected bone surfaces. Therefore, the need exists for systems and methods of bone surface preparation for prosthesis implantation that address the aforementioned problems.

Current methods of bone surface preparation and prosthesis implantation as they relate to ankle joint replacement typically include an anterior to posterior approach and implantation procedure. This procedure suffers from disadvantages known to those of skill in the art relating to, for example, blood supply, boney access, and the amount of bone involved.

### SUMMARY

Ankle prosthesis apparatuses, systems and methods are provided as disclosed herein. Additionally, systems and methods for bone resection and implantation of prosthetics are provided, including surgical techniques and related instrumentation.

An ankle prosthesis apparatus can include a talar component that can be configured as disclosed herein and can have a lower surface with a bone fixation portion for fixation to a talus bone and an upper surface designed for articulation with a bearing component. The bearing component can be configured as disclosed herein and can have a lower surface for articulation with the talar component and an upper surface for articulation with a tibial component. The tibial component can be configured as disclosed herein and can have a lower surface for articulation with the bearing component and an upper surface with a bone fixation portion for fixation to a tibia bone and/or a fibula bone. The bearing component can have a protrusion on its upper surface adapted for engagement with a recess on the tibial component to allow desired rotational and translational movement.

Methods and systems to prepare a bone surface for implantation of a prosthesis can include determining a location for a curved cut line on the bone surface and drilling a series of holes tangent to the curved cut line to create a curved bone resection surface. Methods and systems for the implantation of an ankle prosthesis can include the use of an alignment guide, tibia and talus drill guides, tibia and talus saw guides, and tibia and talus broach guides, all components that can be placed on and removed from a plurality of alignment anchor pins throughout the implantation procedure. A method for medially to laterally implanting an ankle joint prosthesis can include exposing tibia and talus bones from the medial side, resection of the tibia and talus bones, broaching the tibia and talus bones, and positioning and affixing the ankle joint prosthesis components.

It is therefore an object to provide novel ankle prosthesis apparatuses, systems and methods and novel systems and methods for bone resection and prosthetic implantation. An object having been stated hereinabove, and which is achieved in whole or in part by the present subject matter, other objects will become evident as the description proceeds when taken in connection with the accompanying drawings as best described hereinbelow.

### BRIEF DESCRIPTION OF THE DRAWINGS

FIG. 1A of the drawings is a top perspective view of an assembled ankle prosthesis apparatus according to the present disclosure;

FIG. 1B of the drawings is a front end or anterior view of the ankle prosthesis apparatus of FIG. 1;

FIG. 1C of the drawings is a rear end or posterior view of the ankle prosthesis apparatus of FIG. 1;

FIG. 1D of the drawings is a medial side view of the ankle prosthesis apparatus of FIG. 1;

FIG. 1E of the drawings is a lateral side view of the ankle prosthesis apparatus of FIG. 1;

FIG. 1F of the drawings is a top plan view of the ankle prosthesis apparatus of FIG. 1;

FIG. 1G of the drawings is a bottom plan view of the ankle prosthesis apparatus of FIG. 1;

FIG. 1H of the drawings is a front or anterior cross-sectional view of the ankle prosthesis apparatus of FIG. 1 drawn along line H-H of FIG. 1E;

FIG. 2A of the drawings is a top perspective view of the talar component;

FIG. 2B of the drawings is a bottom perspective view of the talar component of FIG. 2A;

FIG. 2C of the drawings is a front end or anterior view of the talar component of FIG. 2A;

FIG. 2D of the drawings is a rear end or posterior view of the talar component of FIG. 2A;

FIG. 2E of the drawings is a lateral side view of the talar component of FIG. 2A;

FIG. 2F of the drawings is a medial side view of the talar component of FIG. 2A;

FIG. 2G of the drawings is a top plan view of the talar component of FIG. 2A;

FIG. 2H of the drawings is a bottom plan view of the talar component of FIG. 2A;

FIG. 2I of the drawings is a schematic diagram of a portion of the talar component of FIG. 2A illustrating its upper surface;

FIG. 3A of the drawings is a top perspective view of the bearing component;

FIG. 3B of the drawings is a bottom perspective view of the bearing component of FIG. 3A;

FIG. 3C of the drawings is a front end or anterior view of the bearing component of FIG. 3A;

FIG. 3D of the drawings is a rear end or posterior view of the bearing component of FIG. 3A;

FIG. 3E of the drawings is a lateral side view of the bearing component of FIG. 3A;

FIG. 3F of the drawings is a medial side view of the bearing component of FIG. 3A;

FIG. 3G of the drawings is a top plan view of the bearing component of FIG. 3A;

FIG. 3H of the drawings is a bottom plan view of the bearing component of FIG. 3A;

FIG. 4A of the drawings is a top perspective view of the tibial component;

FIG. 4B of the drawings is a bottom perspective view of the tibial component of FIG. 4A;

FIG. 4C of the drawings is a front end or anterior view of the tibial component of FIG. 4A;

FIG. 4D of the drawings is a rear end or posterior view of the tibial component of FIG. 4A;

FIG. 4E of the drawings is a lateral side view of the tibial component of FIG. 4A;

FIG. 4F of the drawings is a medial side view of the tibial component of FIG. 4A;

FIG. 4G of the drawings is a top plan view of the tibial component of FIG. 4A;

FIG. 4H of the drawings is a bottom plan view of the tibial component of FIG. 4A;

FIG. 4I of the drawings is a side elevation view of two bone fasteners that could be used with the tibial component;

FIG. 5 of the drawings is a perspective view of an alignment guide, tibia drill guide, and talus drill guide according to the present disclosure;

FIG. 6 of the drawings is a perspective view of a secondary drill guide according to the present disclosure;

FIG. 7A of the drawings is a front end view of a tibia saw guide according to the present disclosure;

FIG. 7B of the drawings is a front end view of a talus saw guide according to the present disclosure;

FIG. 8 of the drawings is a front end view of tibia and talus broach guides according to the present disclosure;

FIG. 9A of the drawings is a perspective view of a talus bone saw;

FIG. 9B of the drawings is a perspective view of a tibia bone saw;

FIG. 10 of the drawings is a lateral elevation view of the bones of the ankle area of a right human foot;

FIG. 11 of the drawings is a perspective view of a method of exposure of the tibia/talus bone interface by resection of the fibula lateral malleolus according to the present disclosure;

FIG. 12 of the drawings is a perspective view of a method of exposure of the tibia/talus bone interface by resection of the tibia medial malleolus according to the present disclosure;

FIG. 13 of the drawings is a perspective view of a method of alignment and placement of the alignment guide, tibia drill guide, and talus drill guide according to the present disclosure;

FIG. 14 of the drawings is a perspective view of a method of depth scouting according to the present disclosure;

FIG. 15 of the drawings is a perspective view of a method of rough resection of bone material using a drilling procedure according to the present disclosure;

FIGS. 16A and 16B of the drawings are perspective views of a method of finish resection of bone material using bone saws according to the present disclosure;

FIGS. 17A-17C of the drawings are perspective views of a method of broaching of the tibia bone according to the present disclosure;

FIG. 18 of the drawings is a perspective view of a method of broaching of the talus bone according to the present disclosure;

FIGS. 19A-19C of the drawings are perspective views of a method of implantation of the tibial prosthesis component according to the present disclosure; and

FIG. 20 of the drawings is a perspective view of a method of implantation of the talar prosthesis component according to the present disclosure.

### DETAILED DESCRIPTION

In accordance with the present disclosure, ankle prosthesis apparatuses, systems and methods are provided. Additionally, systems and methods for bone resection and implantation of prosthetics are provided, including surgical techniques and related instrumentation.

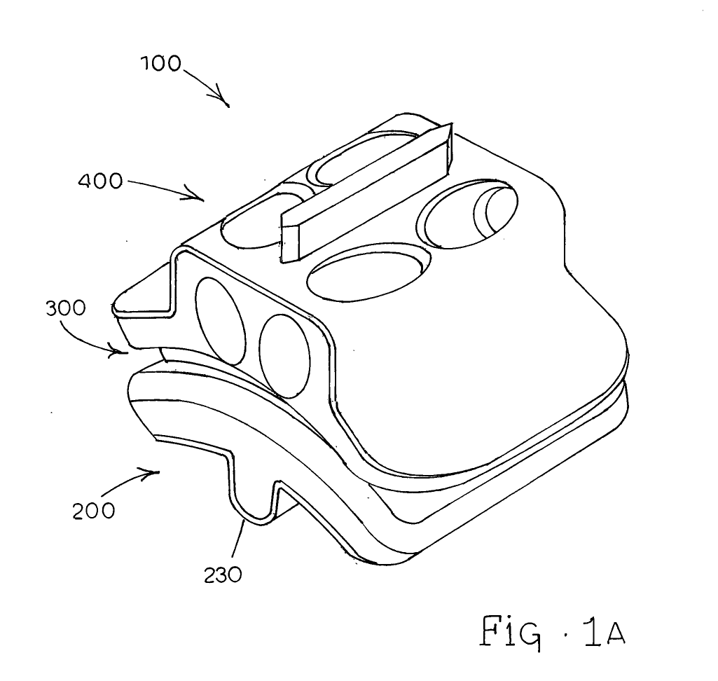

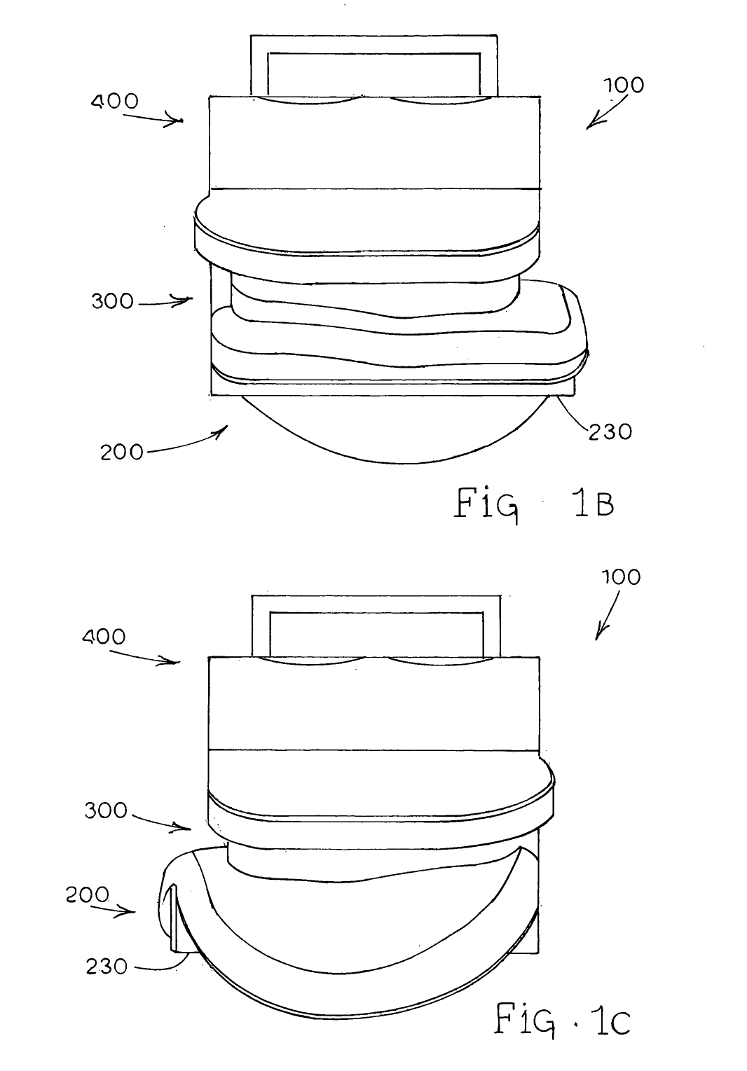

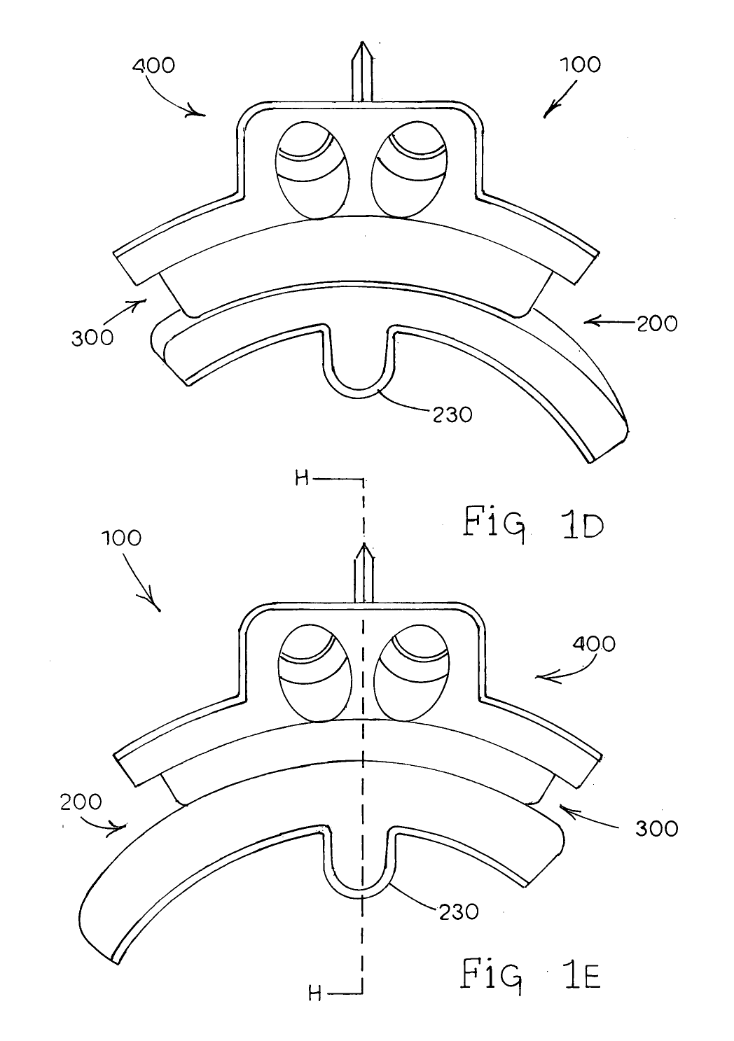

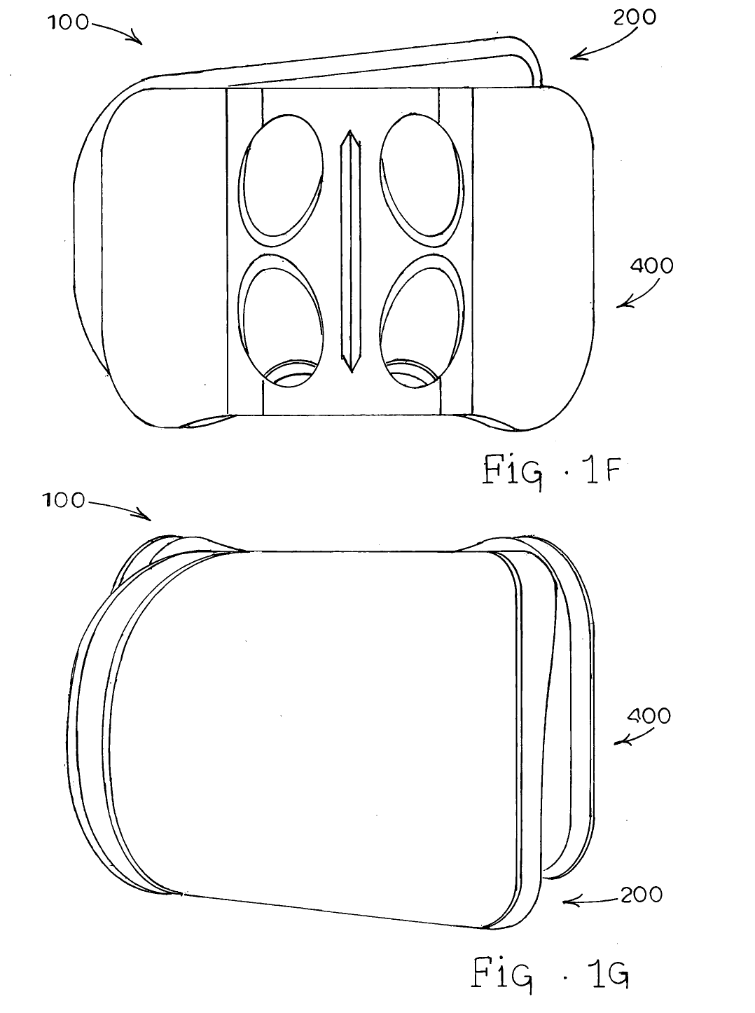

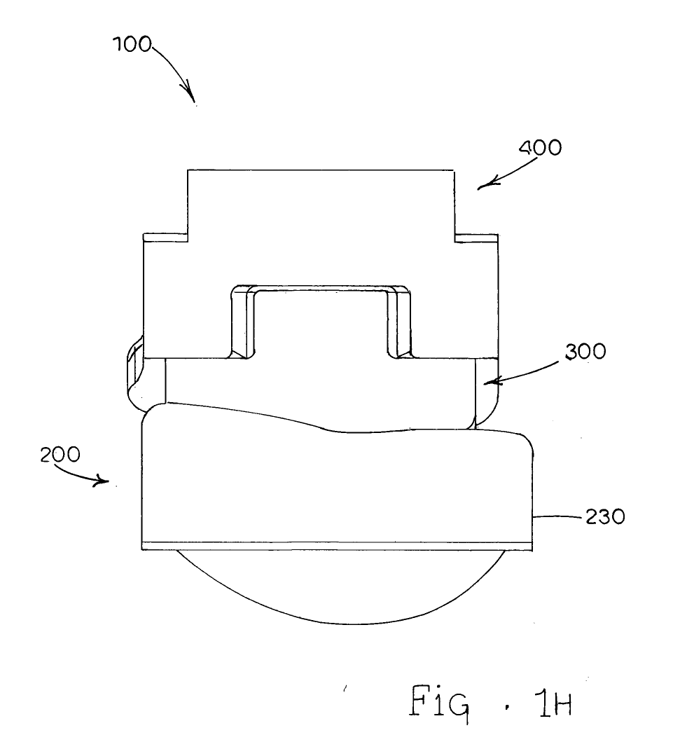

Referring to FIGS. 1A-1H of the drawings, various views of an assembled ankle prosthesis apparatus generally designated 100 are provided. Referring specifically to FIG. 1A of the drawings, a top perspective view of an embodiment of ankle prosthesis apparatus 100 is illustrated. As shown, ankle prosthesis 100 comprises a lower talar component generally designated 200, a bearing component generally designated 300 positioned above and against talar component 200, and an upper tibial component generally designated 400 positioned above and against bearing component 300. FIG. 1B of the drawings provides an anterior view of ankle apparatus 100, and FIG. 1C of the drawings provides a posterior view of ankle apparatus 100. A medial side view of ankle prosthesis apparatus 100 is illustrated in FIG. 1D of the drawings, and a lateral side view of ankle prosthesis apparatus 100 is illustrated in FIG. 1E of the drawings. FIG. 1F of the drawings provides a top plan view of ankle prosthesis apparatus 100, and FIG. 1G of the drawings provides a bottom plan view of ankle prosthesis apparatus 100. Bearing component 300 is hidden from view in FIGS. 1F and 1G. FIG. 1H of the drawings is a front or anterior cross-section view of ankle prosthesis apparatus 100 drawn along line H-H of FIG. 1E of the drawings.

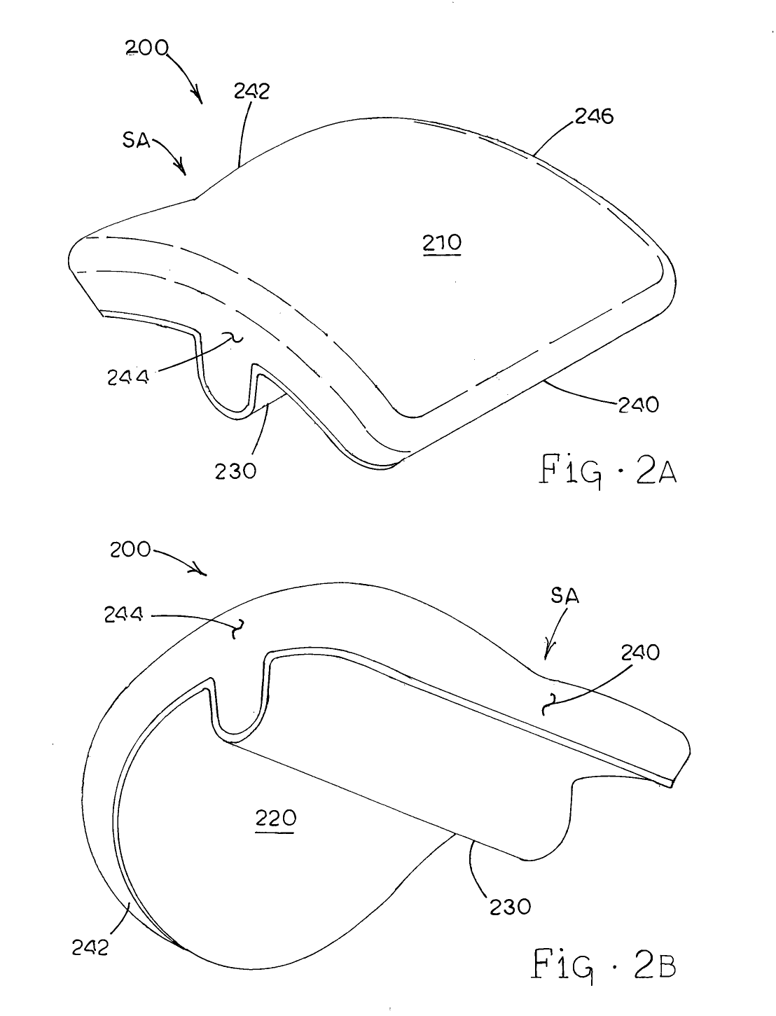

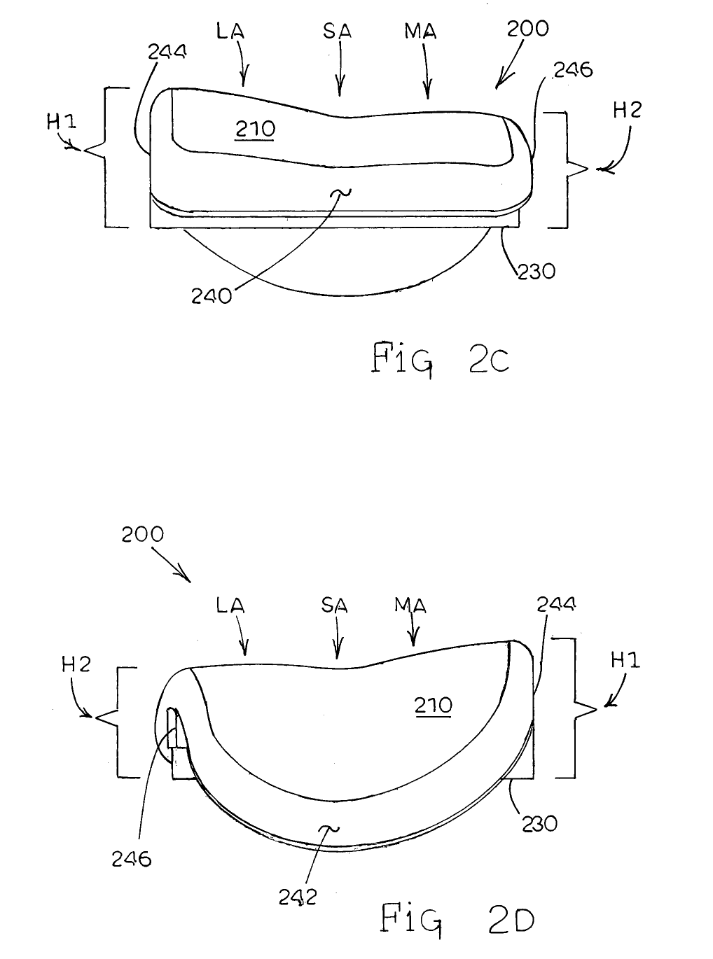

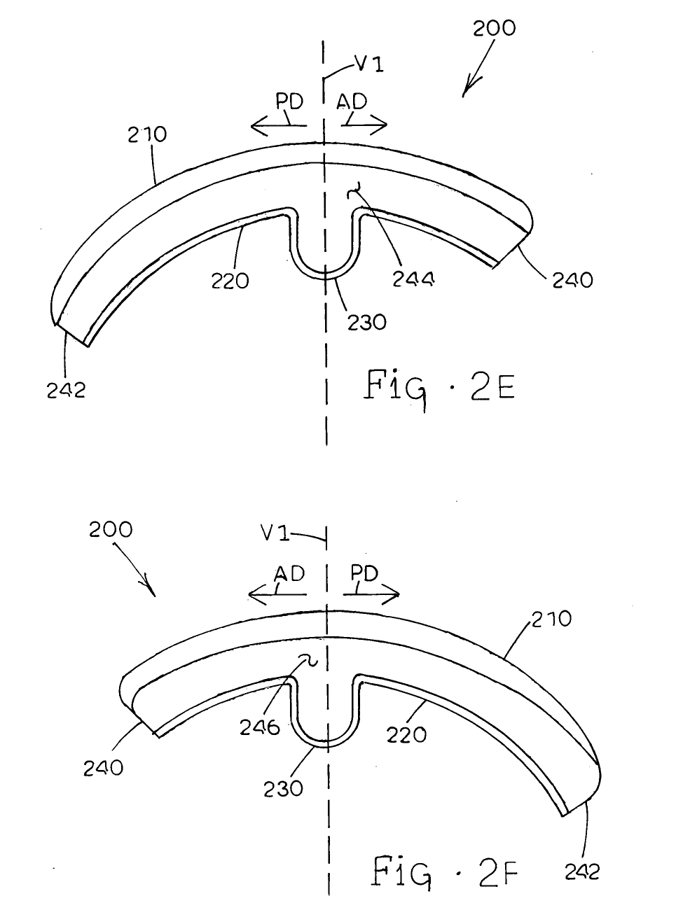

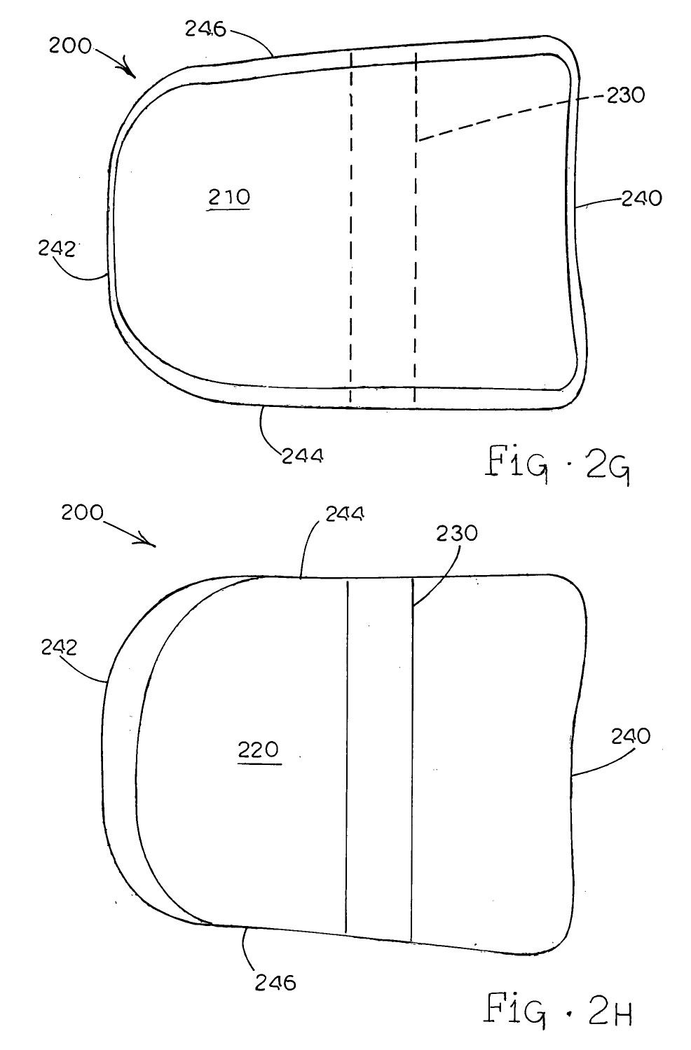

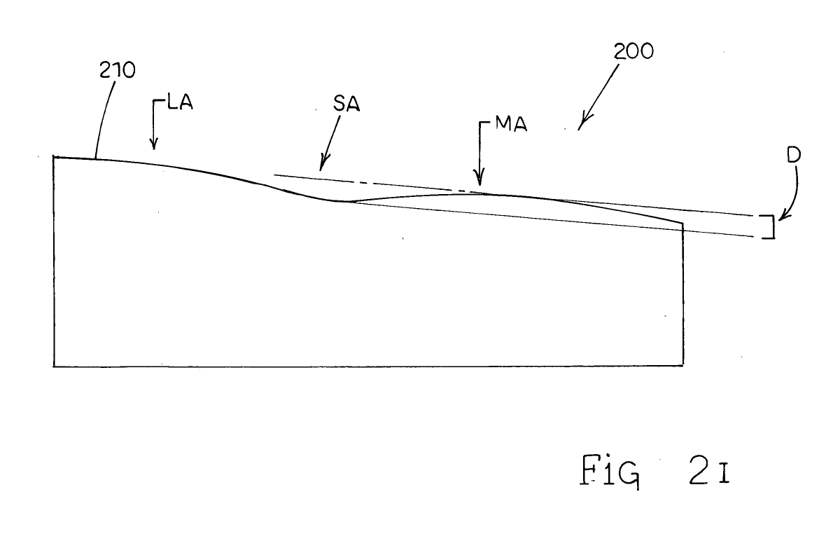

Referring now to FIGS. 2A-2I of the drawings, various views of isolated talar component 200 are provided. A top perspective view of talar component 200 illustrated in FIG. 2A, and a bottom perspective view of talar component 200 is illustrated in FIG. 2B of the drawings. An anterior view of talar component 200 is illustrated in FIG. 2C, and a posterior view of talar component 200 is illustrated in 2D of the drawings. A lateral side view of talar component 200 is illustrated in FIG. 2E, and a medial side view of talar component 200 is illustrated in FIG. 2F of the drawings. A top plan view of talar component 200 is illustrated in FIG. 2G, and a bottom plan view of talar component 200 is illustrated in FIG. 2H. A schematic diagram of a portion of an upper surface of talar component 200 is illustrated in FIG. 2I of the drawings.

Talar component 200 can be made from any suitable material for an ankle prosthesis apparatus such as, for example, a metallic material such as cobalt-chrome or a titanium alloy, or any other biologically stable and suitable material. A titanium plasma spray (TPS) can be applied to desirable surfaces of talar component 200. Talar component 200 is adapted for attachment to a talus bone as further described herein. Referring to FIGS. 2A-2I, talar component 200 can have an upper surface 210 that can have a shape suitable for articulation with bearing component 300. Upper surface 210 of talar component 200 can be curved in any suitable shape for articulation with bearing component 300. As shown, upper surface 210 of talar component 200 is curved at least generally in an arc shape in an anterior to posterior direction and can be at least generally convex. Talar component 200 also can have a lower surface 220 that can be curved like upper surface 210 in an anterior to posterior direction but at least generally concave.

As illustrated in FIGS. 2C and 2D of the drawings, a lateral side height generally designated H1 can be greater than a medial side height generally designated H2 of talar component 200. Lateral side height H1 and medial side height H2 both extend from the bottom of rib 230 to the top side edge of upper surface 210 of talar component 200. Because of the increased lateral side height of talar component 200, upper surface 210 can be sloped in a lateral to medial side direction. In accordance with present disclosure and as shown particularly in FIGS. 2C, 2D and 2I of the drawings, upper surface 210 of talar component 200 can be sloped so as to form a lateral arc generally designated LA and a medial arc generally designated MA. Between lateral arc LA and medial arc MA, a sulcus arc generally designated SA can be formed as an intersection of lateral arc LA and medial arc MA. Sulcus arc SA as positioned and disposed between lateral arc LA and medial arc MA can be a depressed arc region wherein a depth D, shown in FIG. 2I, can exist between the bottom of sulcus arc SA and a straight line drawn across the top surfaces of lateral arc LA and medial arc MA. Sulcus arc SA helps to provide a stabilizing effect between bearing component 300 and talar component 200.

For attachment of talar component 200 to a talus bone, any suitable structure can be utilized on lower surface 220 of talar component 200. As illustrated, talar component 200 can have a bone attachment portion that can be a rib 230 on lower surface 220 to facilitate attachment of talar component 200 to a talus bone. Rib 230 can physically extend on lower surface 220 of talar component 200 at least generally perpendicularly to a vertical axis V1. The extension of rib 230 can be from between a lateral side surface 244 of talar component 200 and an opposite, medial side surface 246 of talar component 200 as shown and as further described below. As shown in FIGS. 2E and 2F of the drawings, rib 230 can be positioned between asymmetrical extensions of talar component 200 on lower surface 220 of talar component 200 such that talar component 200 extends further and is longer on one side of rib 230 than on the opposite side of rib 230. As illustrated in FIGS. 2E and 2F, talar component 200 extends further in a posterior direction PD from vertical axis V1 than in an anterior direction AD from vertical axis V1. The overall shape of talar component 200 can be a shape that can be a portion of a cone that can have an included angle of, for example, 24 degrees. When in this shape, lateral side height H1 and medial side height H2 of talar component 200 can both be separate and different radii from what would be a central axis for the cone.

As illustrated in FIGS. 2G and 2H of the drawings which provide top and bottom plan views, respectively, of talar component 200, anterior side surface 240 can be less curved or straighter than posterior side surface 242 as notable best from a top or bottom view. Additionally, anterior side surface 240 can be longer than posterior side surface 242 as talar component 200 can have a lateral side surface 244 and an opposite, medial side surface 246 that both extend between anterior side surface 240 and posterior side surface 242. As illustrated in FIGS. 2G and 2H, lateral and medial side surfaces 244 and 246 can both taper gradually inwardly as they extend from anterior side surface 240 to posterior side surface 242. Upper surface 210 and lower surface 220 can therefore have a similar, tapered perimeter.

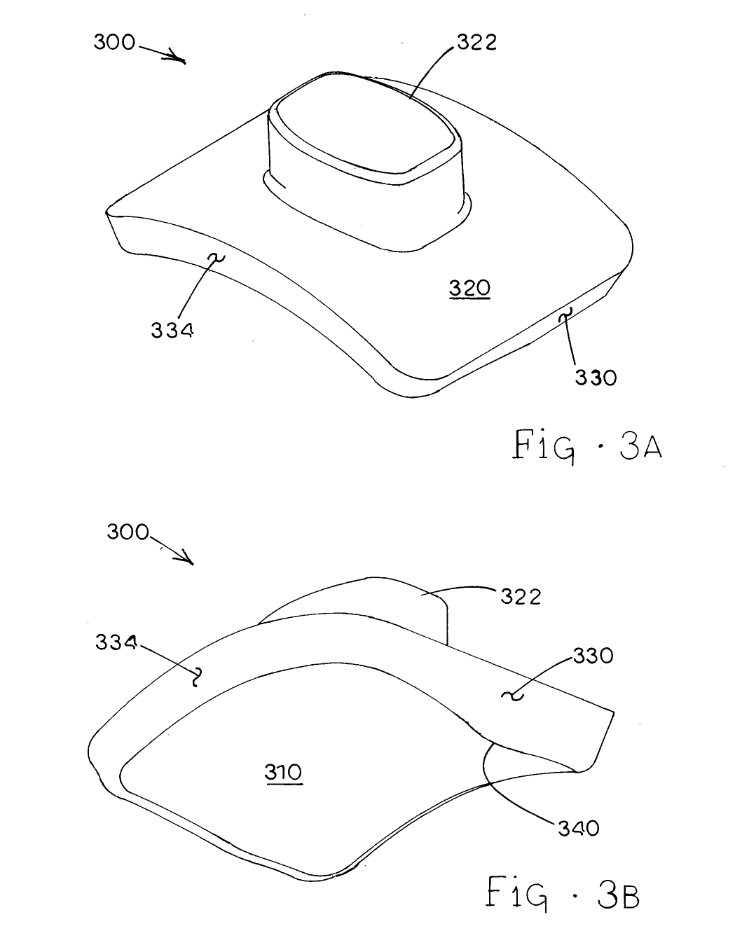

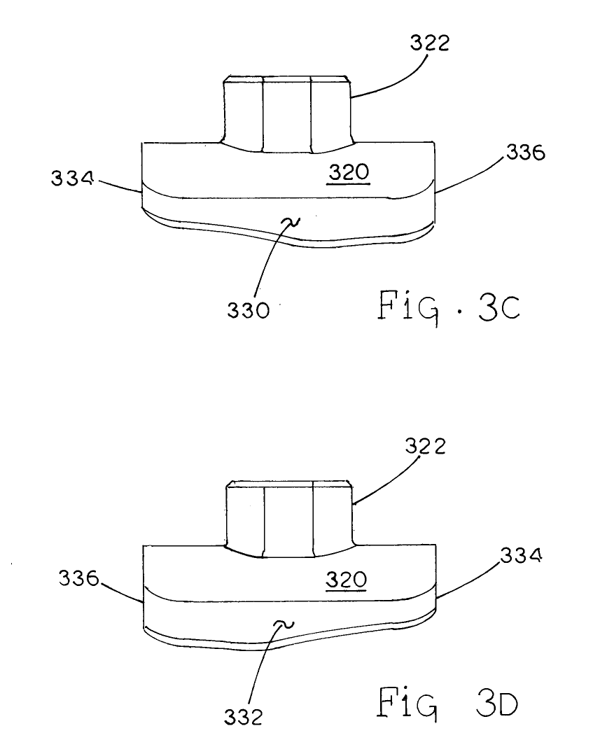

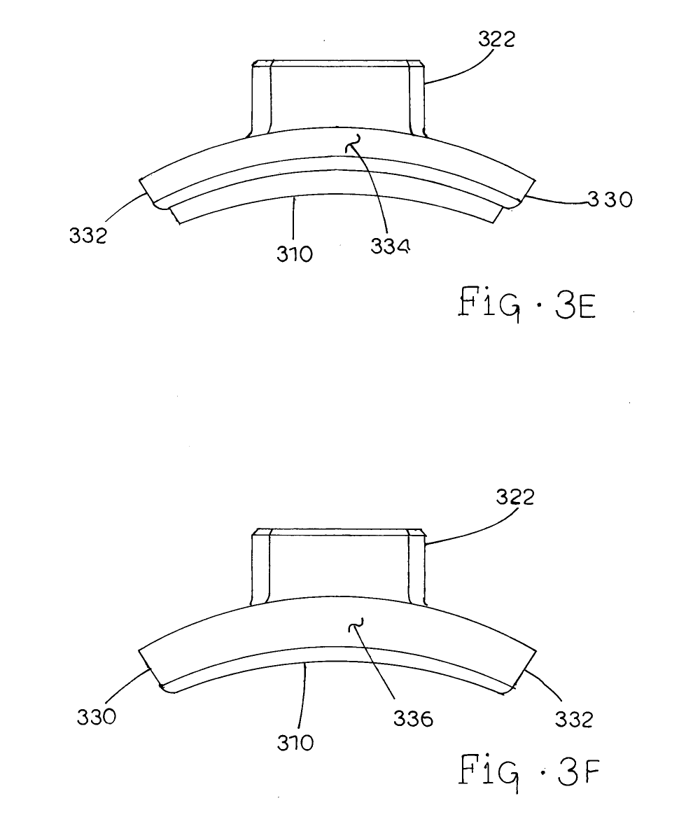

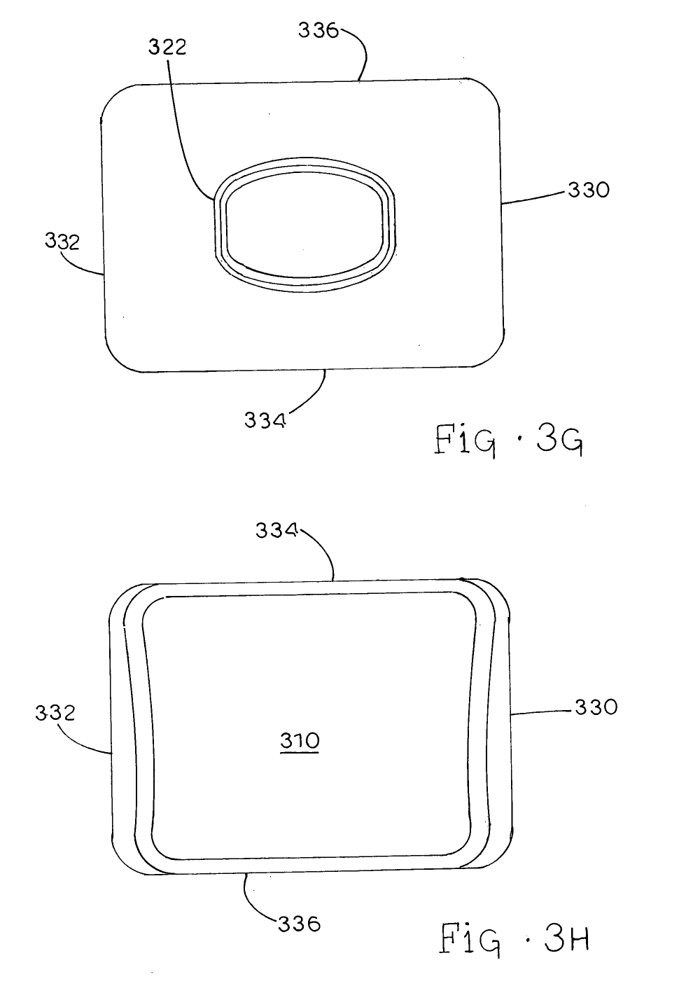

Referring now to FIGS. 3A-3H of the drawings, various views of bearing component 300 are provided. Bearing component 300 can comprise a suitable plastic material such as ultra-high molecular weight polyethylene (UHMWPE) or any other suitable material. Bearing component 300 is designed for placement between talar component 200 and tibial component 400 and can comprise a lower surface 310 for cooperative engagement with upper surface 210 of talar component 200. On an opposite side from lower surface 310, bearing component 300 can include an upper surface 320 that can include at least one protrusion that can be a bearing plug such as bearing plug 322 adapted for cooperative engagement with tibial component 400 as discussed below.

As illustrated, bearing component 300 can have an anterior side surface 330, a posterior side surface 332, a lateral side surface 334 and a medial side surface 336. Lower surface 310 of bearing component 300 can comprise a single radius on a lateral side and two radii on the medial side. The radii on the medial side can be greater, such as by 2 mm, than the corresponding radii on upper surface 210 of talar component 200. The radius on the lateral side of bearing component 300 can be greater, such as by 1 mm, than the corresponding radius on upper surface 210 of talar component 200. Both lower and upper surfaces 310 and 320, respectively, of bearing component 300 can be curved as lower surface 310 can be at least generally concave and upper surface 320 can be at least generally convex. As can be appreciated by those of skill in the art, the various radii of lower surface 310 of bearing component 300 can be changed or altered as desired in order to provide for and allow a desired range of rotational and translational motion for ankle prosthesis apparatus 100. The concavity of lower surface 310 of bearing component 300 can be created by a single, full radius, multiple tangent radii, or constantly varying radii in order to provide for an efficient means of articulation.

Lower surface 310 of bearing component 300 can form a thicker, raised portion 340 that can extend from anterior side surface 330 to posterior side surface 332. Raised portion 340 can be designed for fitting at least substantially into or against sulcus arc SA of talar component 200, and the portions of lower surface 310 of bearing component 300 adjacent to raised portion 340 can be adapted for at least substantially fitting against lateral arc LA and medial arc MA of upper surface 210 of talar component 200. Upper surface 210 of talar component 200 can be formed as described above and shown in the various figures of drawings for upper surface 210 to at least substantially match or matingly engage with lower surface 310 of bearing component 300 in order to provide for a desired range of motion.

As illustrated in FIGS. 3C and 3D of the drawings particularly, lateral side surface 334 of bearing component 300 can have a height less than the height of medial side surface 336 of bearing component 300 to facilitate ankle prosthesis apparatus 100 being anatomically correct.

Bearing plug 322 on upper surface 320 can be of any suitable size and configuration and adapted for fitting against and into a suitably configured recess of tibial component 400. It is envisioned that upper surface 320 of bearing component 300 can include more than one protrusion or bearing plug such as, for example, bearing plug 322, and that tibial component 400 could include any number of suitably configured recesses for fitting against and cooperative engagement with bearing component 300.

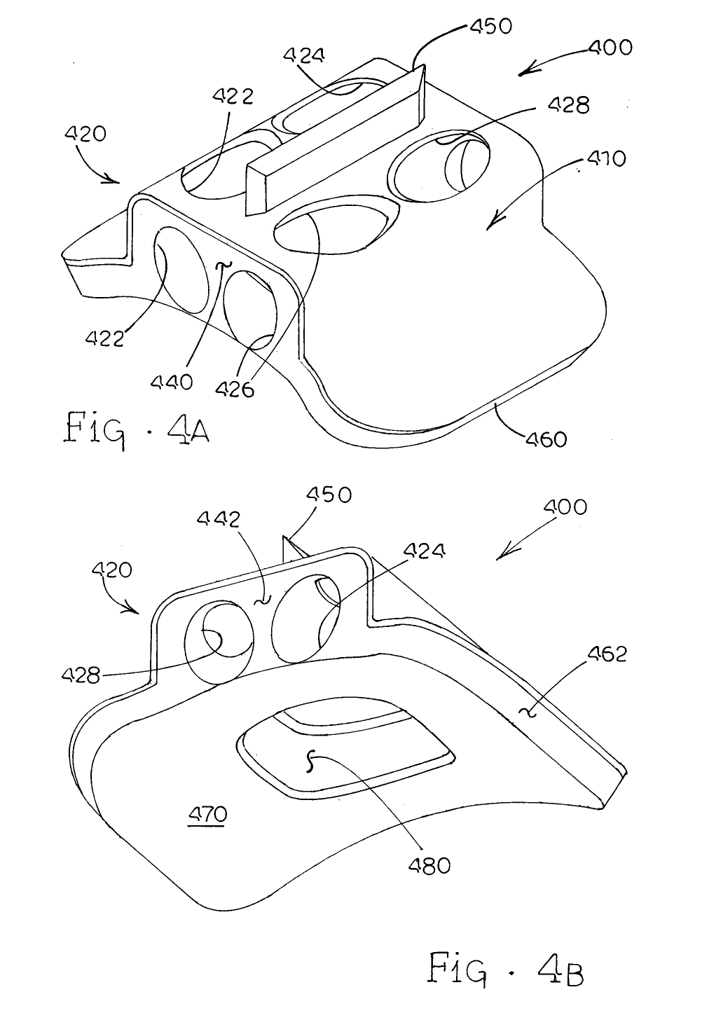

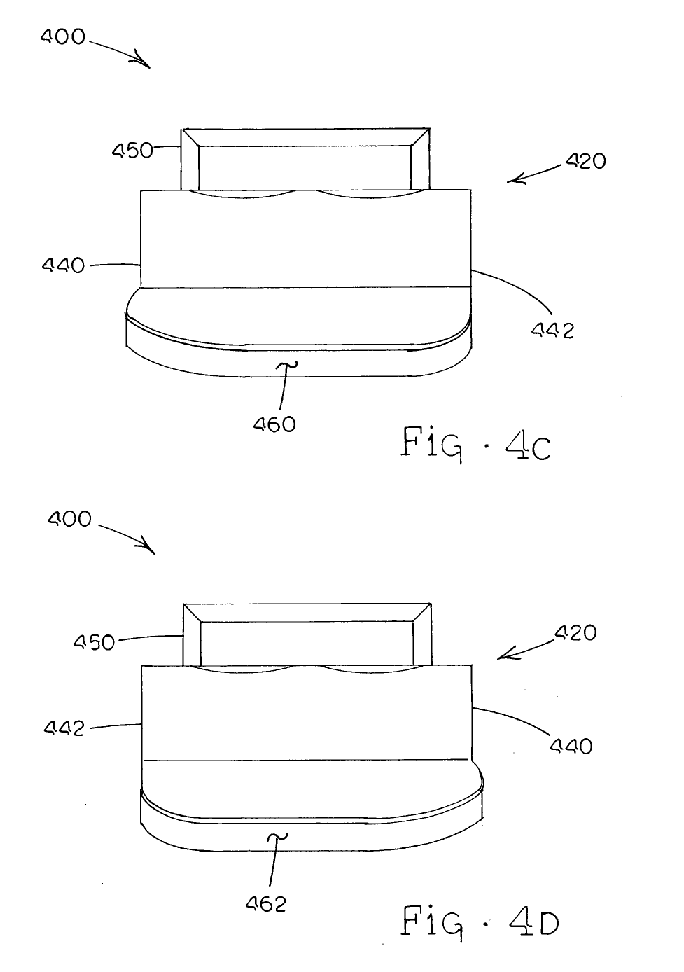

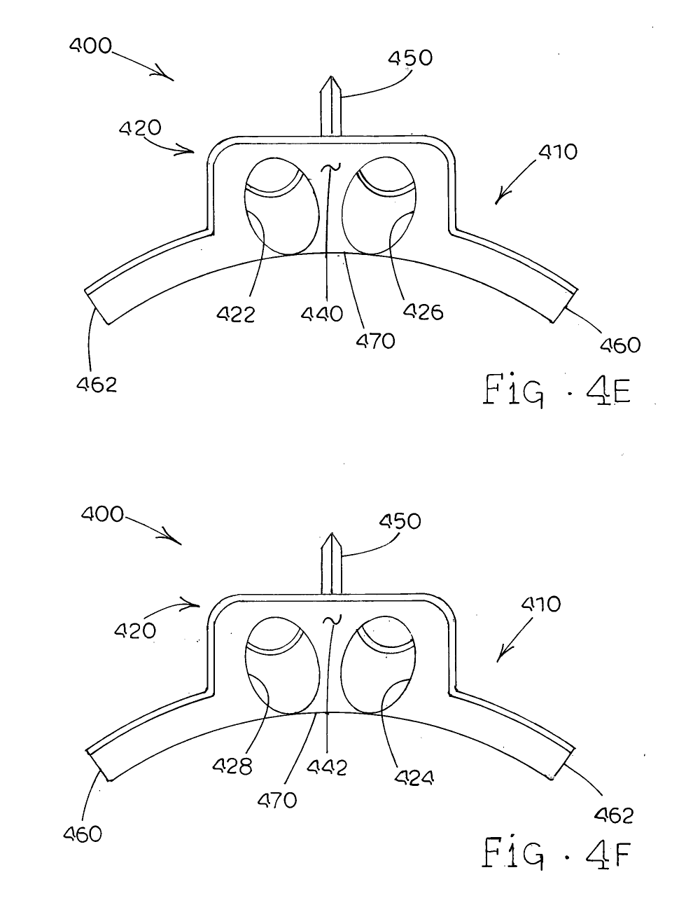

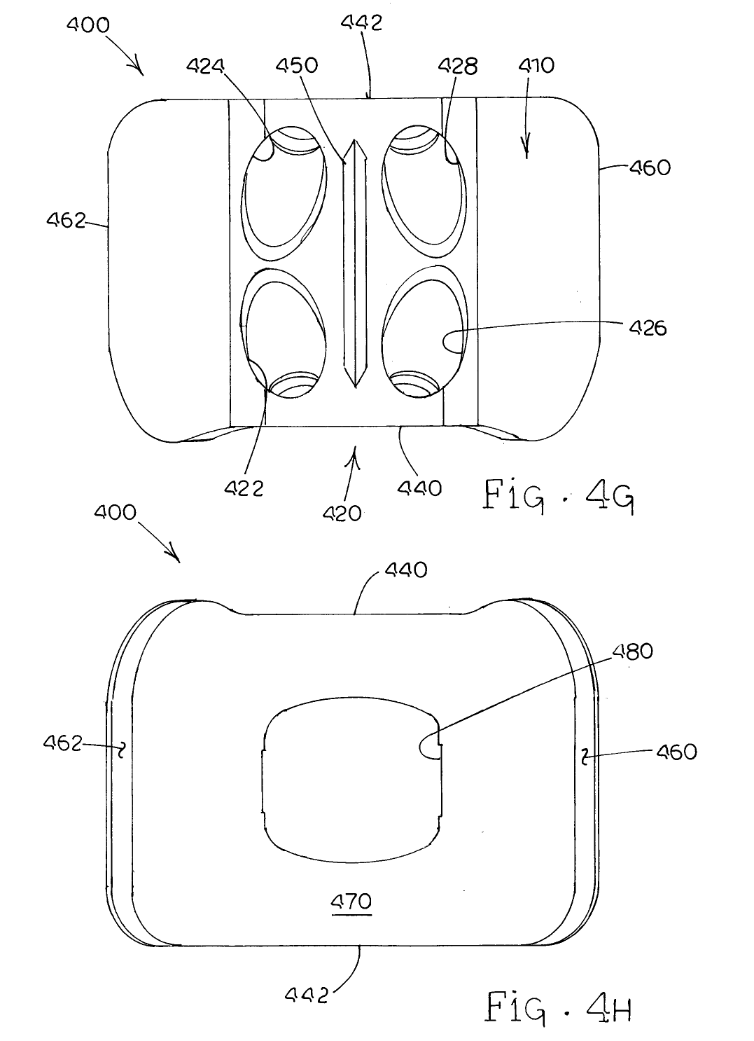

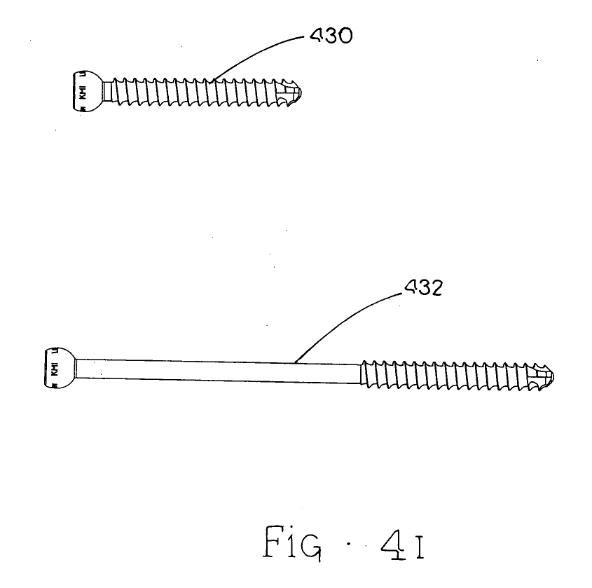

Referring now to FIGS. 4A-4H of the drawings, various views of tibial component 400 are provided. Tibial component 400 can be made from any suitable material, such as from a cobalt-chrome material that can have a titanium plasma spray (TPS) applied to any desired surface, such as to upper surface generally designated 410 of tibial component 400. Upper surface 410 of tibial component 400 can have a tibial attachment portion that can be used to attach tibial component 400 to one or more bones such as to a tibia bone and/or a fibula bone. As shown, the tibial attachment portion can comprise a raised shelf portion generally designated 420 that can define one or more holes such as holes 422, 424, 426 and 428. These holes can receive any suitable type of fastener to achieve initial fixation of tibial component 400 against bone. For example, and as illustrated in FIG. 4I of the drawings, bone screw 430 or lag 432 could be used at least for initial fixation of tibial component 400 against bone. It is envisioned that any other suitable type of bone fastener could be used in accordance with the present disclosure. Non-locking and self-locking fasteners or screws could be used. Each of holes 422-428 can extend through raised shelf 420 diagonally as illustrated particularly in FIGS. 4A, 4B, 4E, 4F and 4G of the drawings wherein one end of each hole can be defined by the top surface of raised shelf 420 and the opposite end of each hole can be defined by a side surface of tibial component 400. For example and as illustrated, holes 422 and 426 extend from the top surface of raised shelf 420 to lateral side surface 440 of tibial component 400. Similarly, holes 424 and 428 can extend from the top surface of raised shelf 420 to medial side surface 442 of tibial component 400.

Upper surface 410 of tibial component 400 can be curved downwardly as tibial component 400 extends on opposite sides of raised shelf 420 away from raised shelf 420. One wing of upper surface 410 and tibial component 400 can extend toward an anterior side surface 460 of tibial component 400, and an opposite wing of upper surface 410 and tibial component 400 can extend toward an opposite, posterior side surface 462 of tibial component 400.

Lower surface 470 of tibial component 400 can be curved and at least generally concave as lower surface 470 can be designed and configured for fitting against upper surface 320 of bearing component 300. A recess 480, as particularly illustrated in FIGS. 4B and 4H of the drawings, can be on lower surface 470 of tibial component 400 and adapted for at least matingly engaging and receiving bearing plug 322 of bearing component 300. As illustrated, recess 480 can be disposed generally centrally on lower surface 470 of tibial component 400, and recess 480 can be of suitable size, shape or configuration as desired and as can be appreciated by those of skill in the art in order to allow for a desired range of motion as tibial component 400 and bearing component 300 interact and articulate with one another. Bearing plug 322 and recess 480 can, for example, both be of a shape that is at least generally square, rectangular of of any other desired and suitable shape as can be appreciated by those of skill in the art. Bearing plug 322 can interface with recess 480 of tibial component 400 in any desirable manner. For example, the interface can occur such that +/−1.5 mm of medial or/lateral translation can occur between bearing component 300 and tibial component 400. Additionally, the interface between bearing plug 322 and recess 480 can be such that +/−5 degrees of axial rotation can occur between bearing component 300 and tibial component 400. The interface between bearing plug 322 and recess 480 of tibial component 400 can allow for +/−0.5 mm of anterior or/posterior translation between bearing component 300 and tibial component 400.

It is envisioned that the present disclosure can further comprise systems for preparing a bone surface for implantation of a prosthesis, systems for preparing a bone surface for implantation of an ankle joint prosthesis, and systems for implanting an ankle joint prosthesis between a patient's distal tibia and talus bones.

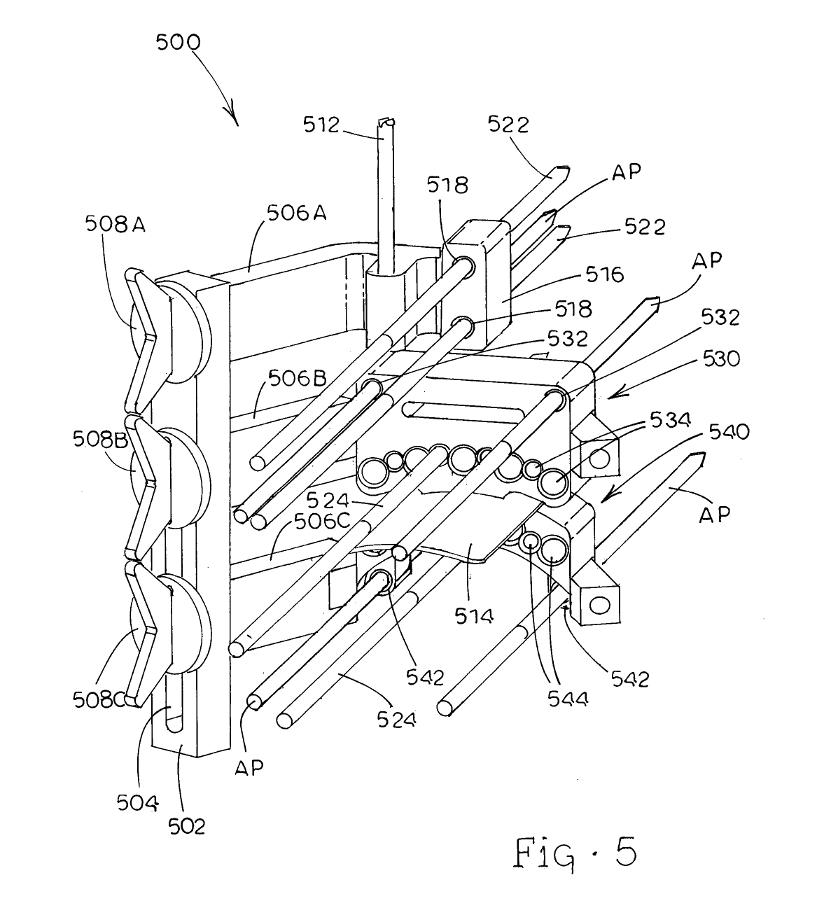

Referring to FIGS. 5 through 9B, a system for implanting an ankle joint prosthesis comprising tibial, talar and bearing components between a patient's distal tibia and talus bones will now be described. It is also understood that fewer of the system components described below may be used for a system for preparing a bone surface for implantation of a prosthesis and a system for preparing a bone surface for implantation of an ankle joint prosthesis.

Referring now to FIG. 5, the preferred system can include an alignment guide, generally designated 500, adapted for alignment of the system with the patient's tibia bone and talus bone. Alignment guide 500 can include a vertical member 502 defining a slot 504 for independent adjustment of attached components as will be described below. A plurality of arms 506A, 506B, 506C are attached to vertical member 502 and are secured thereto by means of adjustment knobs 508A, 508B, 508C, respectively. Alignment guide 500 further includes an alignment rod 512 which can be attached to top arm 506A and is oriented generally parallel to vertical member 502. Alignment guide 500 can further include an alignment tongue 514 which is preferably a thin piece of material capable of placement into the joint between the tibia bone and the talus bone. During installation of an ankle joint prosthesis, proper placement of alignment guide 500 is achieved by using alignment rod 512, which should be parallel with the long axis of the tibia bone, and alignment tongue 514, which is inserted into the joint between the tibia bone and the talus bone for elimination of any anterior/posterior tilting. Alignment guide 500 can further include a fastening section 516 preferably located on the distal end of top arm 506A furthest from vertical member 502 and adjustment knob 508A. Fastening section 516 includes at least one fastening hole 518 through which an appropriate fastener 522, such as for example a 2.0 mm k wire, is threaded for securing of alignment guide 500 to the shaft of the tibia bone.

Referring further to FIG. 5, the system of the present disclosure can further include a tibia drill guide, generally designated 530, which can be attached to alignment guide 500 by way of arm 506B and adjustment knob 508B. Arm 506B and adjustment knob 508B are slidable within slot 504 of vertical member 502 and allow tibia drill guide 530 to be independently adjustable in relation to alignment guide 500. Tibia drill guide 530 includes at least one anchor hole 532 through which an alignment anchor pin AP is threaded for alignment of tibia drill guide 530. Anchor pins AP can also be used for placement and alignment of other system components throughout the prosthesis installation and as will be described further below. Tibia drill guide 530 further defines a plurality of drill holes 534 that are located along a curved path and adapted to receive a drill bit for drilling of a series of holes tangent to a curved cut line. Drill holes 534 can be adjacent to one another and can be alternating in size for different functions throughout the prosthesis installation procedure. For example, several holes may be sized to 2.0 mm for the threading of 2.0 mm scouting k wires 524 for depth readings, whereas adjacent holes can be sized to 3.2 mm for a specific drill bit size in order to resect the bone surface as will be described in further detail below.

Also referring to FIG. 5, a system of the present disclosure can further include a talus drill guide, generally designated 540, for preparation of the talus bone for a prosthesis implant. Talus drill guide 540 can be attached to alignment guide 500 by arm 506C and adjustment knob 508C. As with tibia drill guide 530 described above, talus drill guide 540 can be independently adjustable in relation to alignment guide 500 by arm 506C and adjustment knob 508C, which are slidable within slot 504 of vertical member 502. Talus drill guide 540 can further include at least one anchor hole 542 for the threading of anchor pins AP which, as described with tibia drill guide 530 above, are used for the aligning and guiding of various other components during the implantation procedure. Talus drill guide 540 can further define a plurality of drill holes that are located along a curved path and can receive a drill bit for drilling of a series of holes tangent to a curved cut line. As with the tibia drill guide 530 described above, these holes can be adjacent to one another and can be of varying sizes to accommodate various drill bits and wires for various purposes throughout the implantation procedure as will be described below.

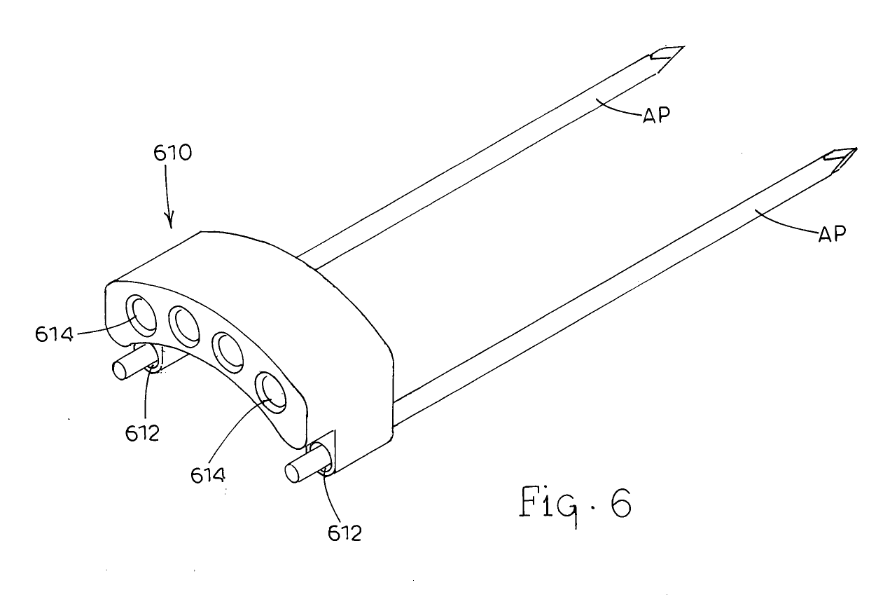

Referring to FIG. 6, the system of the present disclosure can further include a secondary drilling guide such as a talus secondary drilling guide, generally designated 610. Secondary drilling guide 610 can include anchor holes 612 for threading of secondary drilling guide 610 onto previously placed anchor pins AP. Secondary drilling guide can further include a plurality of drill holes 614 which can be located along a curved path and can receive a drill bit for finishing of holes drilled in the tibia or talus bones through tibia drill guide 530 or talus drill guide 540.

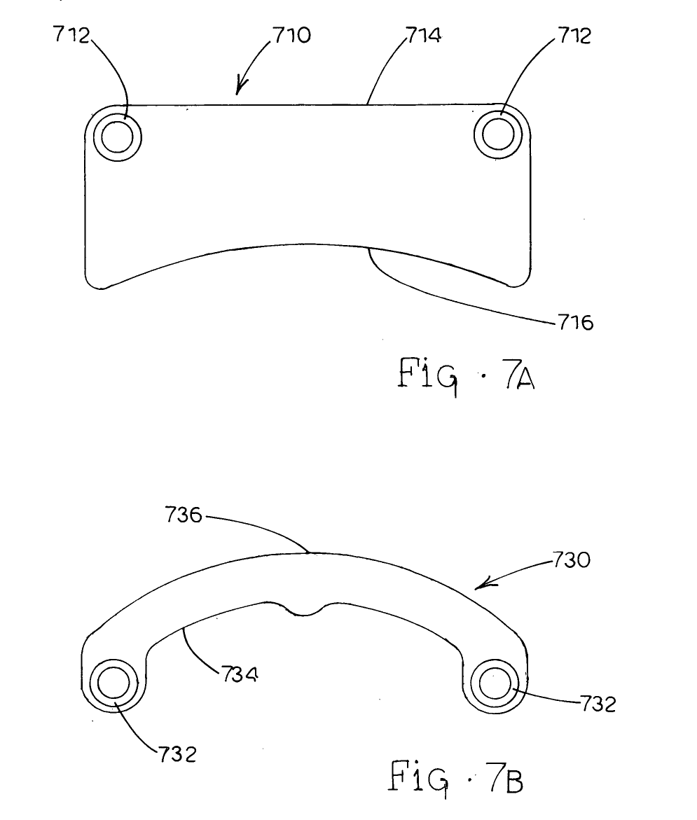

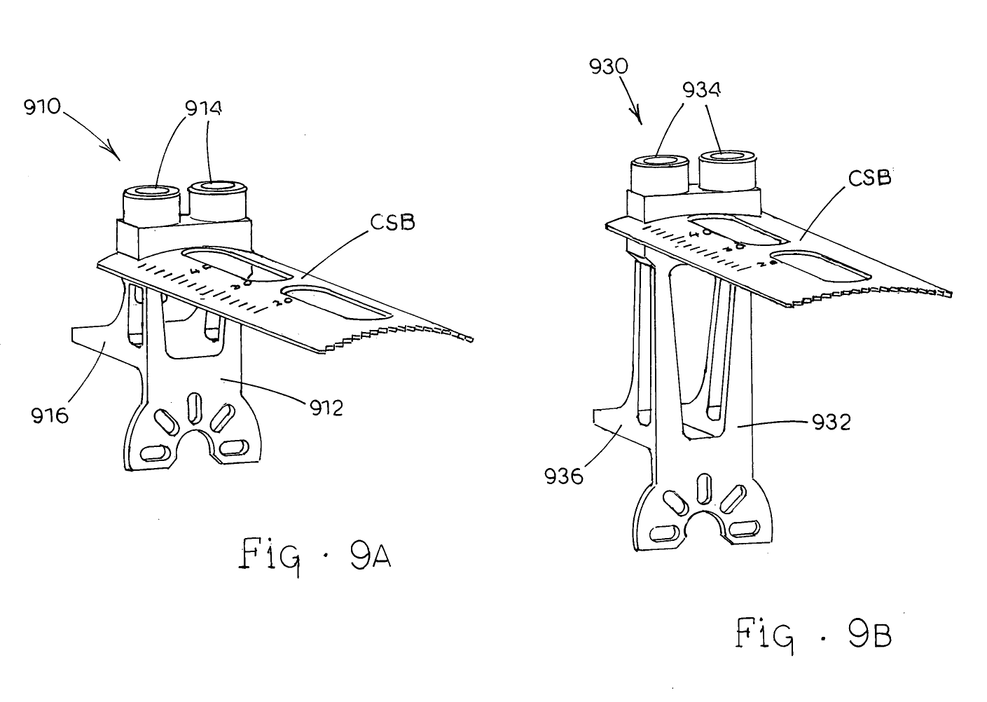

Referring now to FIGS. 7A and 7B, the system of the present disclosure can also include a set of saw guides for the guiding of saw blades for finishing of bone resections as will be described in further detail below. Referring to FIG. 7A, a tibia saw guide, generally designated 710, can be provided for guiding of a saw blade to finish resection of the tibia bone. Tibia saw guide 710 can include anchor holes 712 for the threading of tibia saw guide 710 onto anchor pins AP which are previously inserted into the tibia bone. Tibia saw guide 710 can further include a top surface 714 and a preferably curved bottom saw surface 716. Bottom saw surface 716 is the surface to which a saw blade, preferably a crescentic saw blade (see FIG. 9B) can be guided for finishing of the bone section.

Referring now to FIG. 7B, a talus saw guide, generally designated 730, can also be provided for finishing of a resection cut of the talus bone. Talus saw guide 730 can include anchor holes 732 through which talus saw guide 730 is threaded onto alignment anchor pins AP which have previously been secured within the talus bone. Talus saw guide 730 further includes a bottom surface 734 and a preferably curved top saw surface 736. Top saw surface 736 is capable of guiding a preferably crescentic saw blade (see FIG. 9A) for finishing of a resection cut of the talus bone as will be described in further detail below.

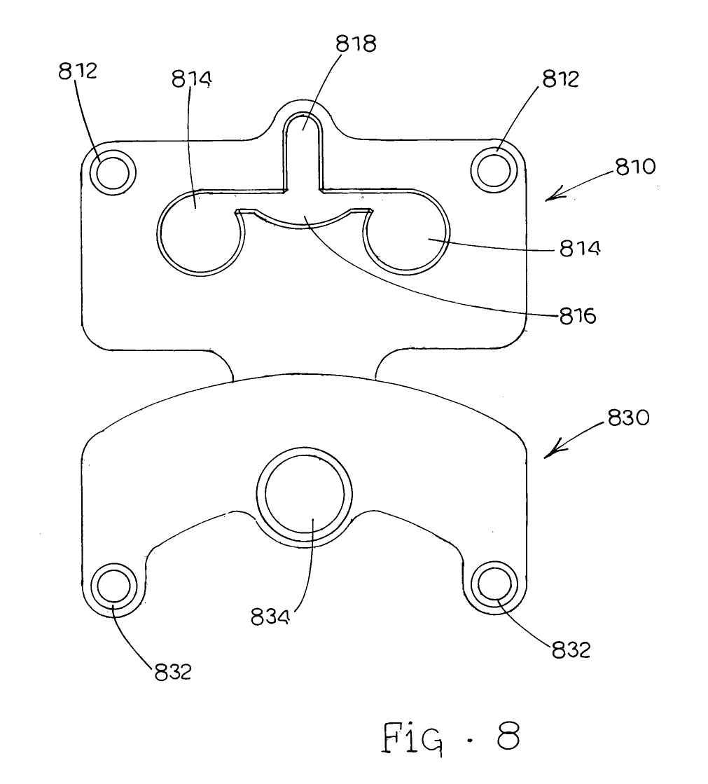

Referring now to FIG. 8, the system of the present disclosure can further include tibia and talus broach guides, generally designated 810 and 830, respectively, for the broaching of the tibia and talus bones after resection and before implantation of the prosthesis components. Tibia broach guide 810 can include anchor holes 812 for the threading of tibia broach guide 810 onto anchor pins AP that have previously been inserted into the tibia bone. In order to create a broach within the tibia bone that matches the profile of a tibial prosthesis component, tibia broach guide 810 can include broach drill holes 814 and a broach bridge 816 which can connect two broach drill holes 814 for the removal of a portion of the resected tibia bone. Additionally, in order to match the top surface profile of a preferable tibial component 400, a broach recess 818 can be defined within tibia broach guide 810 for the guiding of a drill to provide a recess in the tibia bone for matching with a rib on tibial component 400. This recess will assist in fixation and stabilization of tibial component 400 to the tibia bone.

Referring further to FIG. 8, talus broach guide 830 can include anchor holes 832 for the threading of talus broach guide 830 onto anchor pins AP which have previously been secured within a talus bone. Talus broach guide 830 can further include a recess drill hole 834 which can receive a drill bit to drill a recess within the top surface of a resected talus bone to match the profile of a talar prosthesis component. For example, the preferred talar component 200 of the present disclosure comprises a rib disposed on its lower surface and the recess provided on the resected surface of the talus bone by drilling through recess drill hole 834 defined by talus broach guide 830 will allow talar component 200 to be properly affixed to the talus bone.

Referring now to FIGS. 9A and 9B, bone saws that can be used with the system of the present disclosure for finish resection of the talus and tibia bones are shown generally as 910 and 930, respectively. Talus bone saw 910 can include a support member 912 and a handle 916 for gripping by a mechanical handpiece. A preferably crescentic saw blade CSB can be attached to support member 912 and tightened by way of fasteners 914, such as screws. Likewise, tibia bone saw 930 can include a support member 932 and handle 936. Tibia bone saw 930 can also include a preferably crescentic saw blade CSB that is attached to support member 932 by way of fasteners 934.

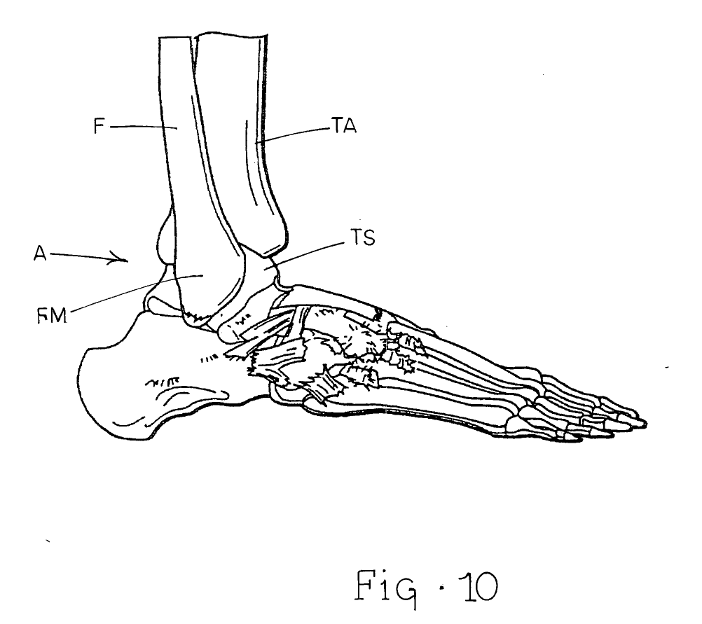

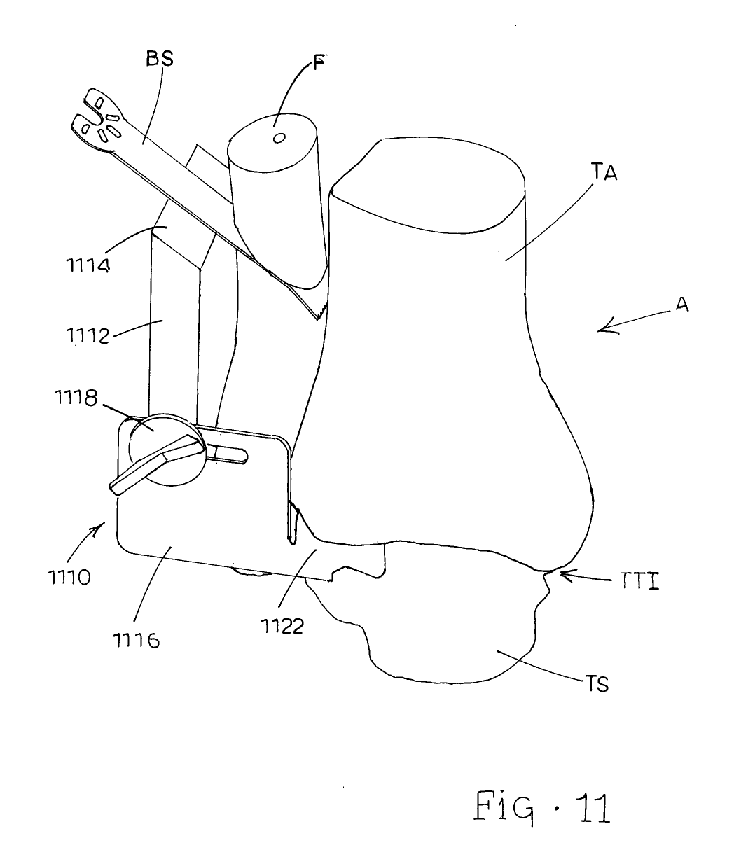

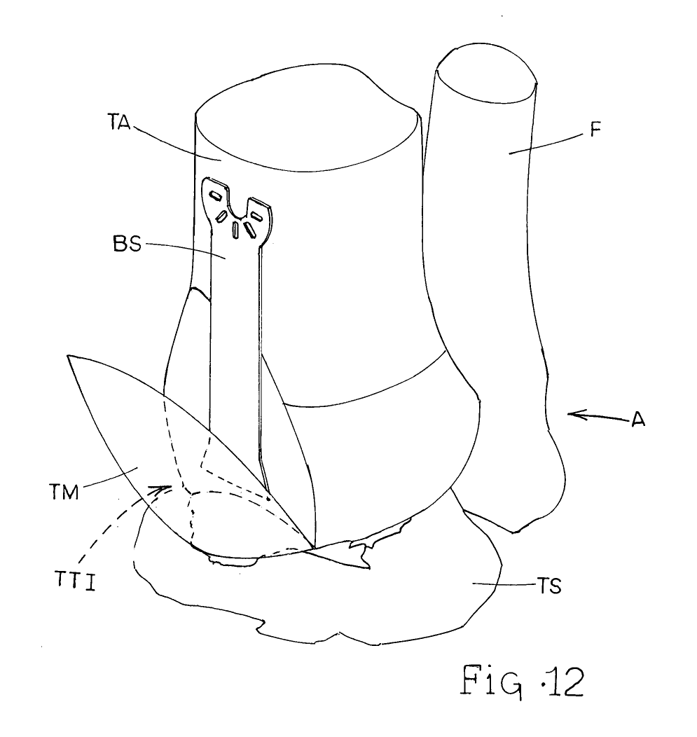

The present disclosure can further include methods of preparing a bone surface for implantation of a prosthesis, methods of preparing a bone surface for implantation of an ankle joint prosthesis, and methods of implanting an ankle joint prosthesis between a patient's distal tibia and talus bones. Referring now to FIGS. 10-20, progressive steps that can be associated with preparation of bone surfaces for implantation of a prosthesis and implanting of an ankle joint prosthesis between a patients' distal tibia and talus bones are illustrated. These steps are illustrated and described herein for exemplary purposes and are not meant to be exhaustive of those which could be taken in preparation of bone surfaces for implantation of a prosthesis and implanting of an ankle joint prosthesis.

Referring to FIG. 10, a lateral, elevation view of a right human foot and ankle area, generally designated A, is shown. The foot and leg bones described with reference to the methods below include a fibula bone F and an associated fibula lateral malleolus FM, a tibia bone TA and an associated tibia medial malleolus (see FIG. 12), and a talus bone TS.

While it is understood that the methods of the present disclosure can include the preparation of any bone surface for implantation of a prosthesis, the following description with reference to FIGS. 11-15B is in reference to the preparation of tibia bone TA and talus bone TS for implantation of an ankle joint prosthesis.

Referring to FIG. 11, the first step in this preparation method can be the exposure of the tibia/talus bone interface, generally designated TTI. While the exposure of tibia/talus bone interface TTI and subsequent prosthesis implantation can be performed laterally to medially, it is understood that this exposure and implantation can also be performed medially to laterally, as will be described further below. To begin the exposure of tibia/talus bone interface TTI, a surgeon can first make an incision on the lateral side of the involved limb near ankle joint area A. This incision can be made high enough in order to resect fibula F for exposure of tibia/talus bone interface TTI. Once the incision is made, a fibula resection guide, generally designated 1110, can be placed in an abutting relationship with fibula F. Fibula resection guide 1110 can include a vertical member 1112 comprising a saw guide face 1114, which typically is oriented at 45° and to which a bone saw BS is guided for making the fibula resection cut. Vertical member 1112 can be attached to an alignment member 1116 by an adjustment knob 1118, which can allow fibula resection guide 1110 to be adjusted for either the left or right side of the body. Alignment member 1116 can further include a tongue portion 1122, which can assist in aligning and placing fibula resection guide 1110 in a correct position. Once fibula resection guide 1110 is placed against fibula bone F, tongue portion 1122 can be placed into tibia/talus bone interface TTI for proper alignment of fibula resection guide 1110. Once fibula resection guide 1110 is properly placed, the surgeon can use bone saw BS for resection of fibula bone F preferably at a 45° angle, which facilitates the reconstruction of fibula bone F once the ankle prosthesis components are inserted. Once fibula bone F has been resected, the distal end of fibula bone F can be rotated in an inferior/posterior direction in order to make room for the guides used during the procedure and to fully expose tibia/talus bone interface TTI.

While the exposure of tibia/talus bone interface TTI described above is performed laterally to medially, it is additionally understood that the exposure of tibia/talus bone interface TTI and subsequent prosthesis implantation can be performed by the methods of the present disclosure in a medially to laterally oriented procedure. With reference to FIG. 12, in the initial tibia/talus bone interface TTI procedure, a surgeon would expose ankle joint area A by making an incision on the medial side of the involved limb. This incision would need to be made high enough in order to resect tibia medial malleolus TM such that tibia/talus bone interface TTI is exposed. In order to accomplish the resection of tibia medial malleolus TM, the surgeon can utilize a bone saw BS to properly resect the required portion of tibia medial malleolus TM wherein tibia medial malleolus TM can be rotated in an inferior direction in order to make room for the guides and instruments for the prosthesis implantation and to provide exposure of tibia/talus bone interface TTI.

Once tibia/talus bone interface TTI is exposed, locations and shapes can be determined for a tibia resection line and a talus resection line for resection of tibia bone TA and talus bone TS, respectively. While the location and shape of the resection lines described below refer to a curved cut line, it is understood that the location and shape of the resection lines of the present disclosure can be of any linear or nonlinear configuration or a combination thereof.

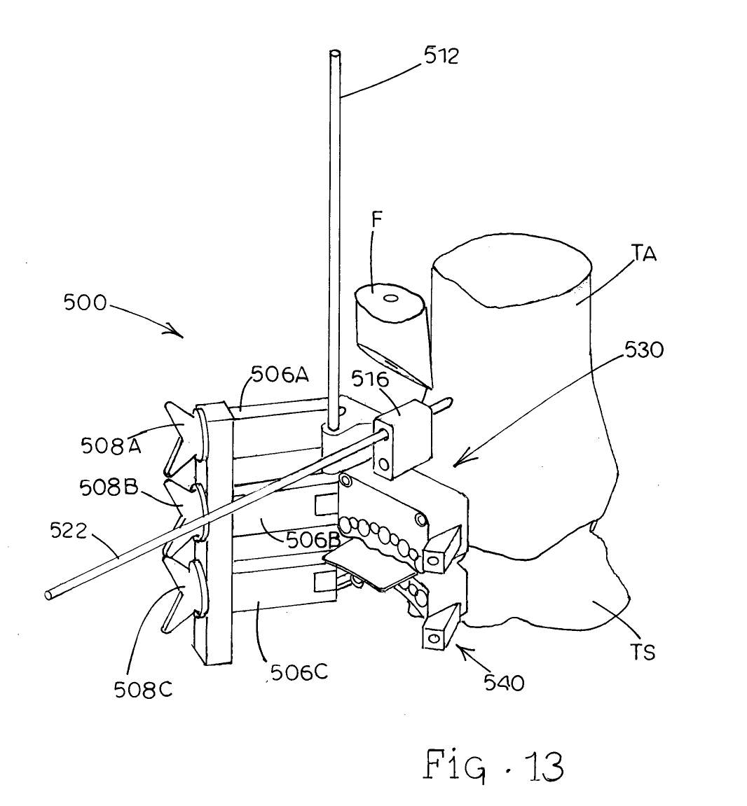

Once the locations and shapes for resection lines on tibia bone TA and talus bone TS have been determined, a properly sized alignment guide 500, tibia drill guide 530, and talus drill guide 540 can be placed for resection of tibia bone TA and talus bone TS for prosthesis implantation. With reference to FIGS. 5 and 13, and as described above, proper placement of alignment guide 500 can be achieved by using alignment rod 512 and alignment tongue 514. Alignment rod 512 can be parallel with the long axis of tibia bone TA and alignment tongue 514 can be inserted into tibia/talus bone interface TTI to reduce anterior/posterior tilt and to help ensure alignment of alignment guide 500, tibia drill guide 530, and talus drill guide 540 such that they are not rotated around a medial/lateral axis. When alignment of alignment guide 500 is established, an appropriate fastener 522, such as a 2.0 mm Steinmann pin, can be placed through one of fastening holes 518 located on fastening section 516 wherein fastener 522 will be secured into tibia bone TA perpendicular to the long axis of tibia bone TA. Once the surgeon confirms that the initial placement of fastener 522 is parallel to the desired cut, another fastener 522 can be placed through an additional fastening hole 518 of alignment guide 500.

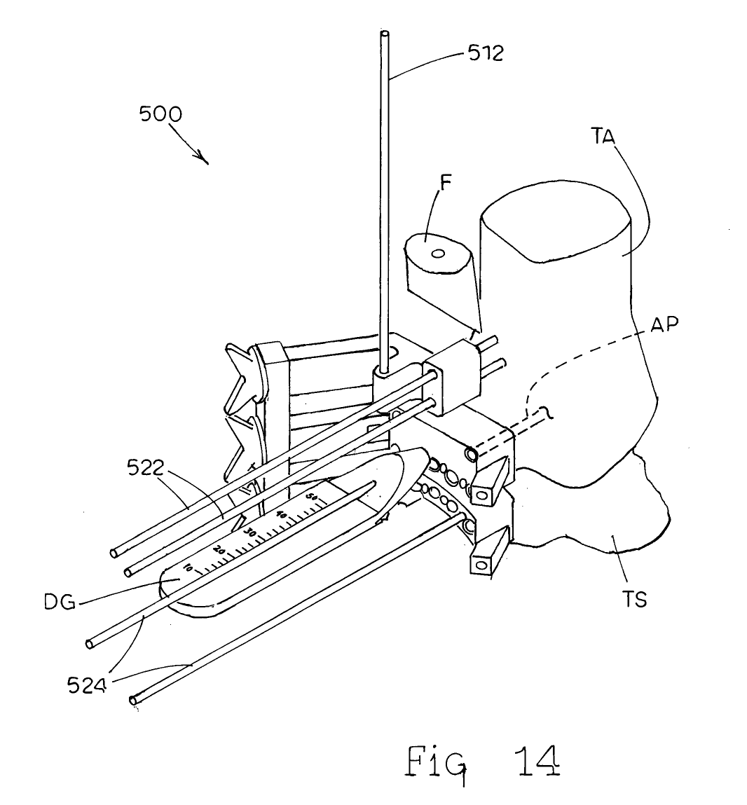

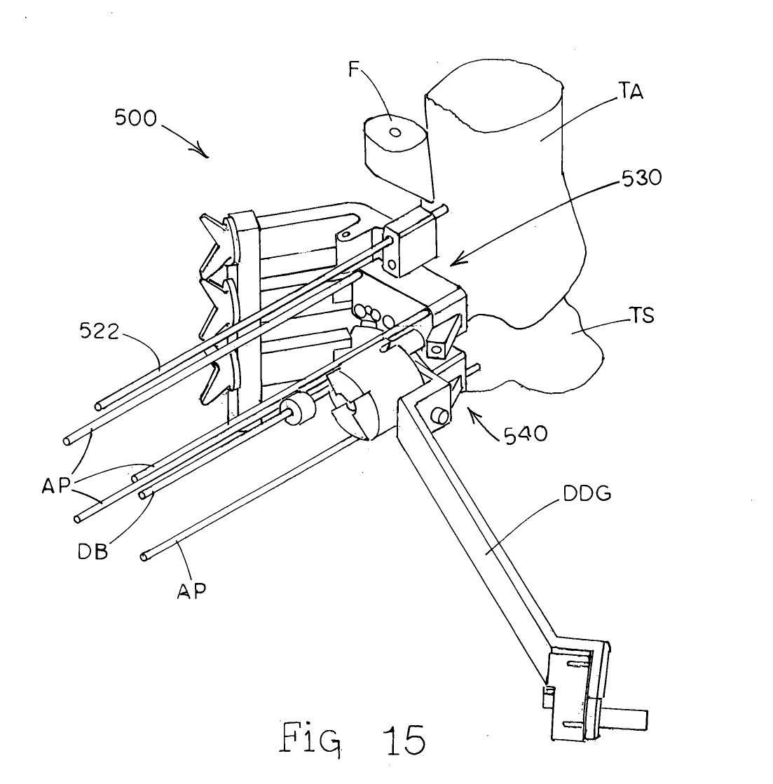

When alignment guide 500 is secure, tibia drill guide 530 and talus drill guide 540 can be adjusted by movements of arms 506B, 506C and adjustment knobs 508B, 508C, respectively, such that tibia drill guide 530 and talus drill guide 540 are independently adjusted in relation to alignment guide 500. Once tibia drill guide 530 and talus drill guide 540 are touching each other, the amount of tibia bone TA and talus bone TS to be resected will be exactly the amount of bone that the prosthesis will replace. Referring to FIGS. 14 and 15, the surgeon can use scouting wires 524 and a depth gauge DG to determine that the direction of the cuts to be made is acceptable for both tibia bone TA and talus bone TS and to help ensure that tibia drill guide 530 and talus drill guide 540 are aligned correctly. Once this confirmation has been made, anchor pins AP should be placed through anchor holes 532 of tibia drill guide 530 and anchor holes 542 of talus drill guide 540 (see FIG. 5). Anchor pins AP can remain in place throughout the implant procedure and can assist in aligning and guiding of other components of the implant system.

Once alignment guide 500 and attached tibia drill guide 530 and talus drill guide 540 have been properly aligned and anchor pins AP have been secured, depth readings can be made by the surgeon through a scouting procedure to determine the depth of cuts on tibia bone TA and talus bone TS for proper resection. Referring to FIG. 14, the surgeon can use scouting wire 524, such as a 2.0 mm Steinmann pin, placed through one of tibia drill holes 534 located on tibia drill guide 530 and one of talus drill holes 544 located on talus drill guide 540. The leading edge of scouting wire 524 should be stopped at the depth of the cut that is desired on tibia bone TA and talus bone TS and the associated depth reading can be made using an appropriate depth gauge DG. Once the proper depth recordings have been made, scouting wires 524 and depth gauge DG can be removed so that the resection drilling cut can be made.

Referring now to FIG. 15, tibia drill guide 530 and talus drill guide 540 can be used to drill holes in tibia bone TA and talus bone TS tangent to a pre-identified curved cut line in order to create a rough resection of the bones. For the resection of tibia bone TA, the surgeon can use appropriate sized drill bits DB to pass through drill holes 534 of tibia drill guide 530 to drill out sections of tibia bone TA wherein the series of holes that are drilled are tangent to a pre-identified tibia curved cut line for resection of the bone. As shown in FIG. 15 (with reference to FIG. 5) the holes drilled through drill holes 534 of tibia drill guide 530 can be adjacent to one another and can be alternating in size, as shown with the 2.0 mm and 3.2 mm alternating drill holes 534 shown in FIG. 5. Additionally, the surgeon preferably can use a drill depth guide DDG to ensure that the depth of the drill holes in tibia bone TA do not exceed the depth readings obtained during the depth scouting procedure discussed above.

Once all drill holes have been made along the curved cut line of tibia bone TA, the same procedure can be used for talus bone TS wherein a drill bit DB is passed through drill holes 544 of talus drill guide 540 to drill a series of holes tangent to a pre-identified talus curved cut line for resection of the bone. As with the resection of tibia bone TA discussed above (and with reference to FIG. 5), the holes drilled through drill holes 544 of talus drill guide 540 can be adjacent to one another and can be alternating in size, as shown with the 2.0 mm and 3.2 mm alternating drill holes 544 shown in FIG. 5. Again, the surgeon preferably can use a drill depth guide DDG to ensure that the depth of the drill holes in talus bone TS do not exceed the depth readings obtained during the depth scouting procedure discussed above.

Once the drilled portions of tibia and talus bones TA, TS are removed, curved bone resection surfaces will remain. At this point, the surgeon can remove alignment guide 500 and attached tibia drill guide 530 and talus drill guide 540 from anchor alignment pins AP. Anchor pins AP can remain secured within tibia bone TA and talus bone TS for use with other components of the system described below.

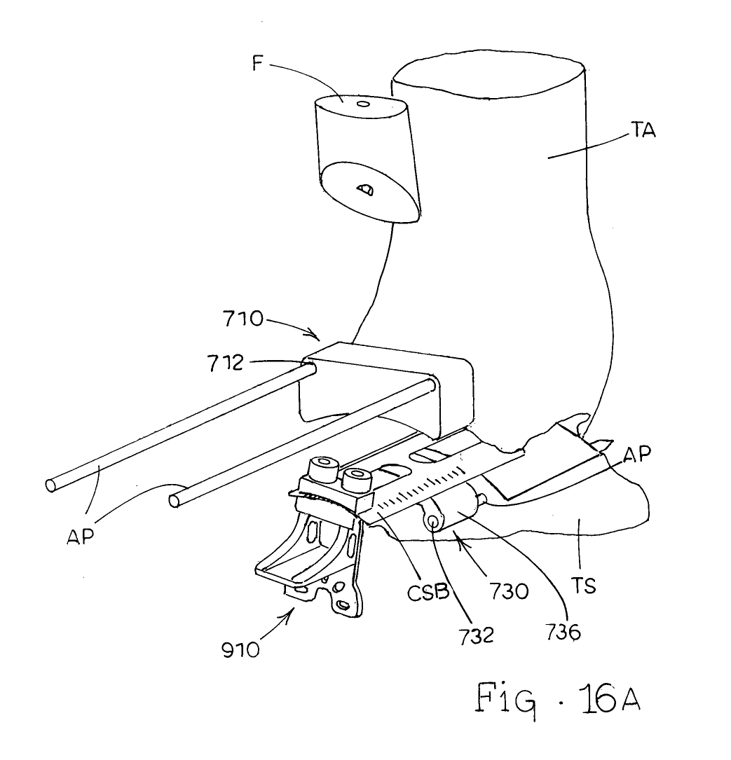

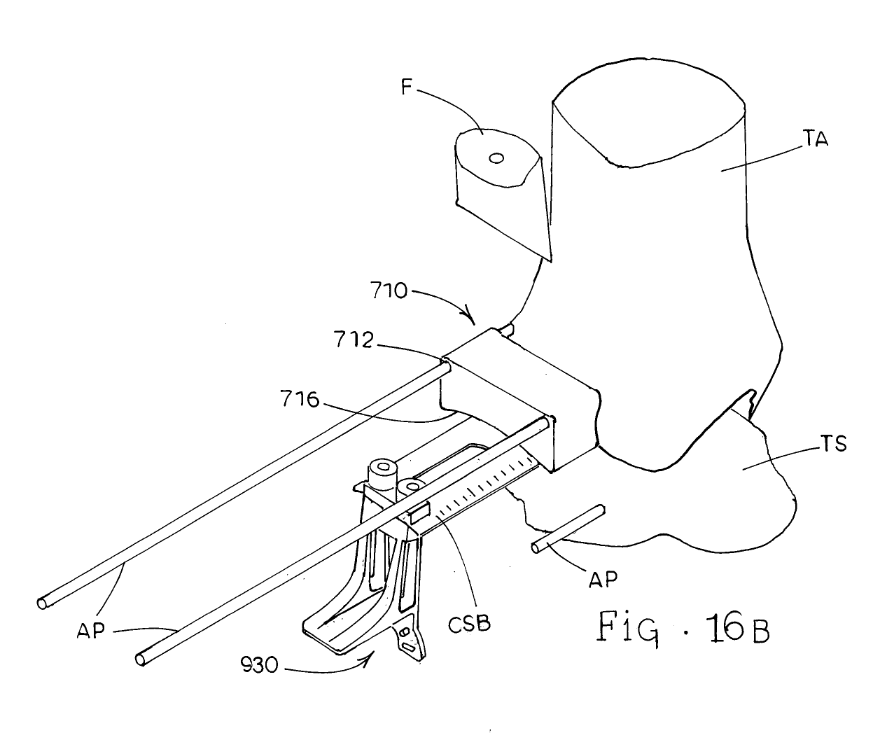

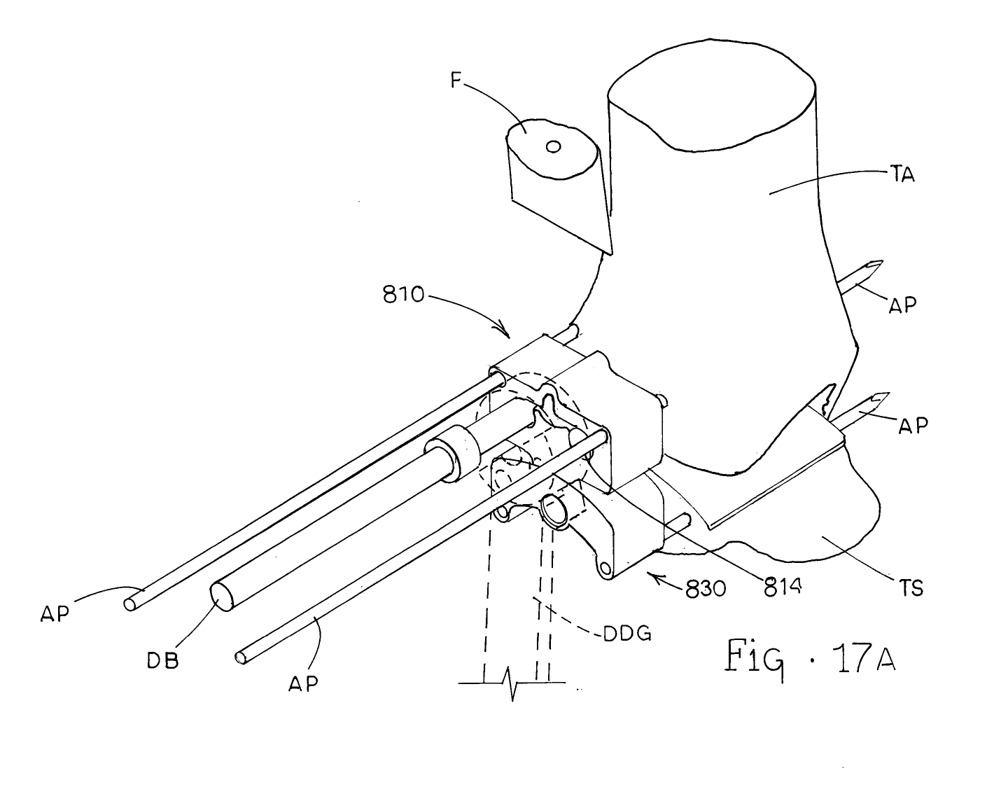

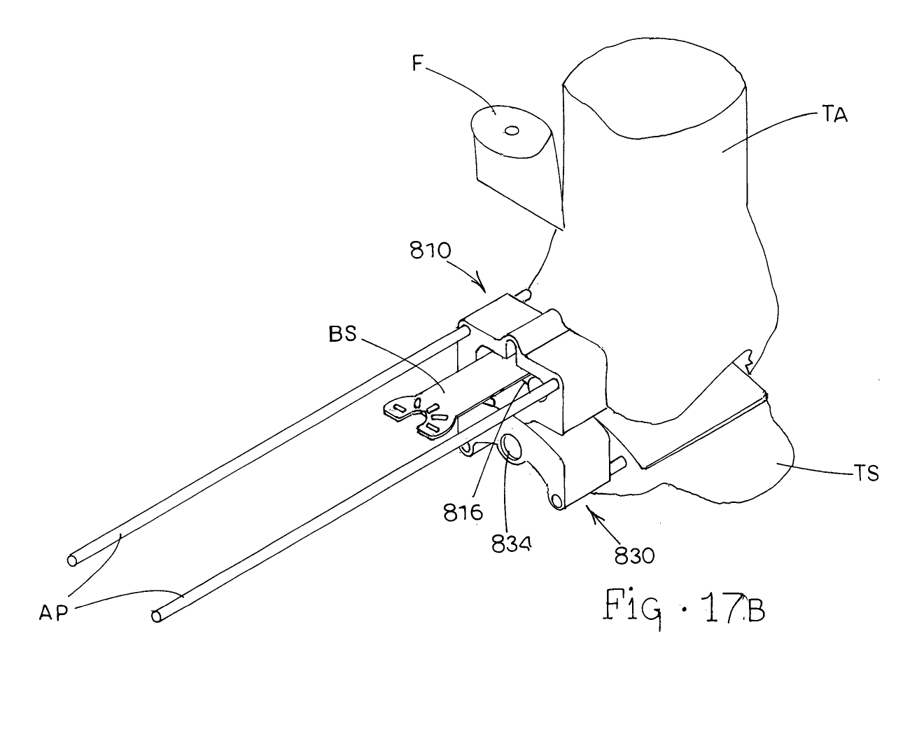

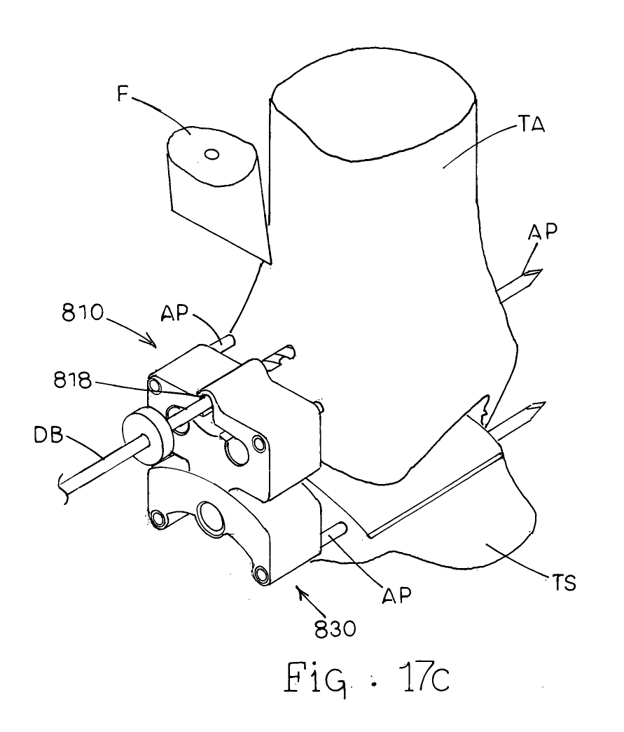

Once the resection surfaces of tibia bone TA and talus bone TS have been roughed in by the drilling procedure described above, a final finishing step can be performed to finish the resection surfaces to create the necessary interface between the bones and the matching prosthesis components. It is understood that finishing of the resection surfaces can be performed by any suitable mechanical or automatic process or apparatus known now or later including manual cutting or laser cutting. As an example and with reference to FIGS. 16A and 16B, tibia saw guide 710 and talus saw guide 730 can be placed over appropriate anchor pins AP through anchor holes 712, 732, respectively, until they abut tibia bone TA and talus bone TS, respectively. Tibia saw guide 710 and talus saw guide 730 are used to guide preferably crescentic saw blade CSB for finishing of the resection surfaces. Crescentic saw blade CSB can be a kerfed and oscillating blade having laser marked gradations thereupon, wherein the depth of cut readings from the depth scouting procedures described above can be used, along with the laser markings, to determine where to stop the depth of the crescentic cut.

As shown in FIG. 16A, the finishing resection of talus bone TS can be accomplished using talus bone saw 910. The surgeon places a saw blade, preferably crescentic saw blade CSB of talus bone saw 910, onto top saw surface 736 of talus saw guide 730. Using the depth readings obtained from the depth scouting procedure discussed above, along with the laser markings on crescentic saw blade CSB, the surgeon can cut and finish the resection surface of talus bone TS to the proper depth. Talus saw guide 730 can then be removed from anchor pins AP while anchor pins AP remain affixed in talus bone TS for later use.

Likewise, and with reference to FIG. 16B, preferably crescentic saw blade CSB of tibia bone saw 930 can be placed underneath bottom saw surface 716 of tibia saw guide 710 for the finishing resection of tibia bone TA. Again, crescentic saw blade CSB of tibia bone saw 930 will have laser markings which can be used in conjunction with the depth readings obtained during depth scouting as described above to help the surgeon determine where to stop the cut. After the finishing resection cut has been made, tibia saw guide 710 can be removed from anchor pins AP while anchor pins AP remained affixed for later use.

Once the finished resection of tibia bone TA and talus bone TS has been completed, resected tibia bone TA and talus bone TS can be broached and the resected surfaces prepared to match the profile of the corresponding prosthesis components. Tibia broach guide 810 and talus broach guide 830 can be placed over anchor pins AP through anchor holes 812, 832, respectively, until they abut tibia bone TA and talus bone TS, respectively.

Referring to FIGS. 17A-17C, the broaching of tibia bone TA using tibia broach guide 810 is preferably performed in three steps once tibia broach guide 810 is placed on anchor pins AP. First, with reference to FIG. 17A, an appropriate sized drill bit DB, for example a 5.0 mm drill bit, is used to drill through broach drill holes 814 of tibia broach guide 810 (see also FIG. 8). Drill depth guide DDG (shown in phantom) can be used to ensure that the proper drill depth is maintained. Next, with reference to FIG. 17B, the surgeon will use bone saw BS to pass through broach bridge 816 to connect the holes drilled in tibia bone TA through broach drill holes 814 (see also FIG. 8). Finally, with reference to FIG. 17C, a drill bit DB, such as a 2.0 mm drill bit, can be passed through broach recess 818 of tibia broach guide 810 in order to create a recess on the resected surface of tibia bone TA to match a rib profile of preferred tibial component 400. If necessary, the surgeon can finish any of the rough cuts through the use of a fine bone saw (not shown). Tibia broach guide 810 can then be removed from anchor pins AP.

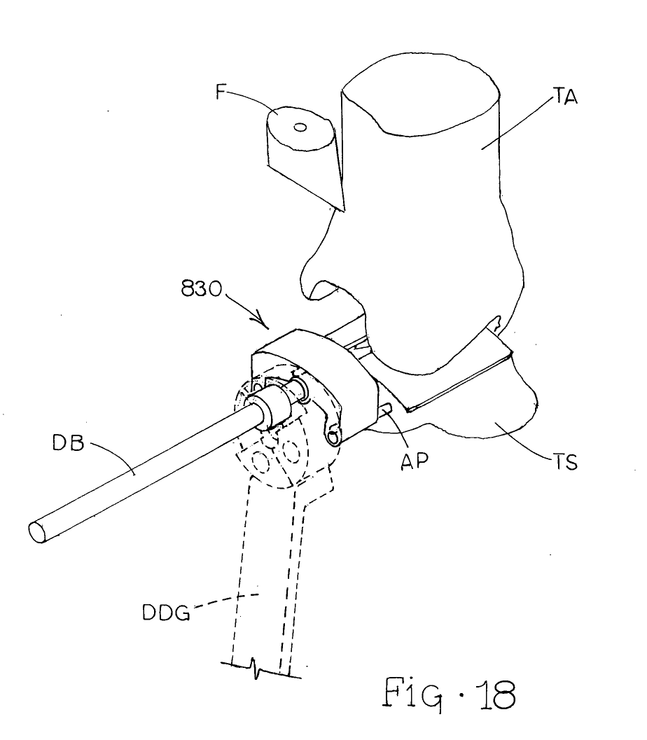

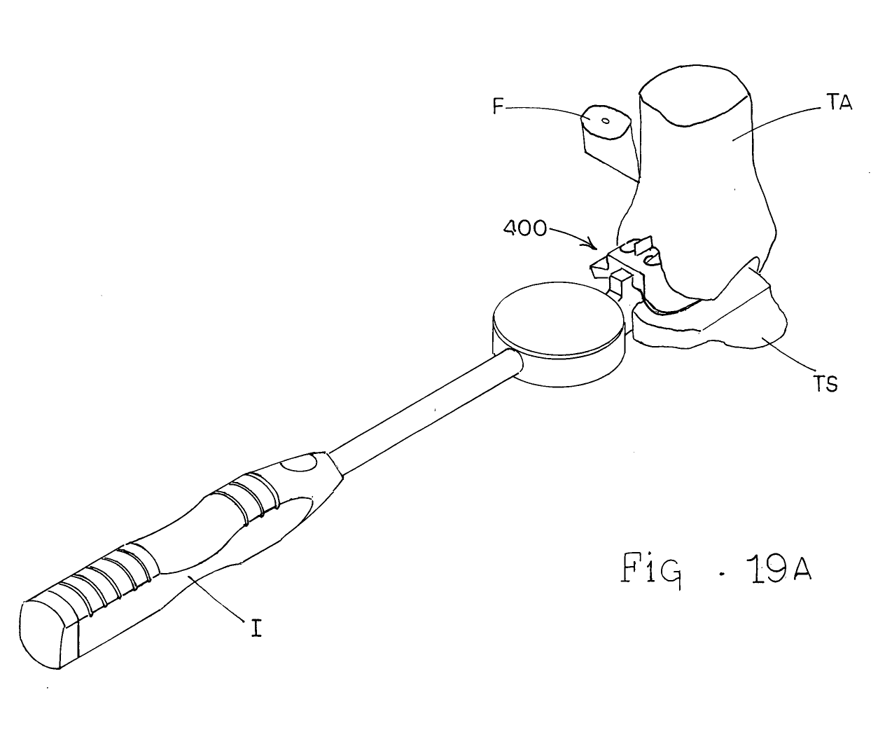

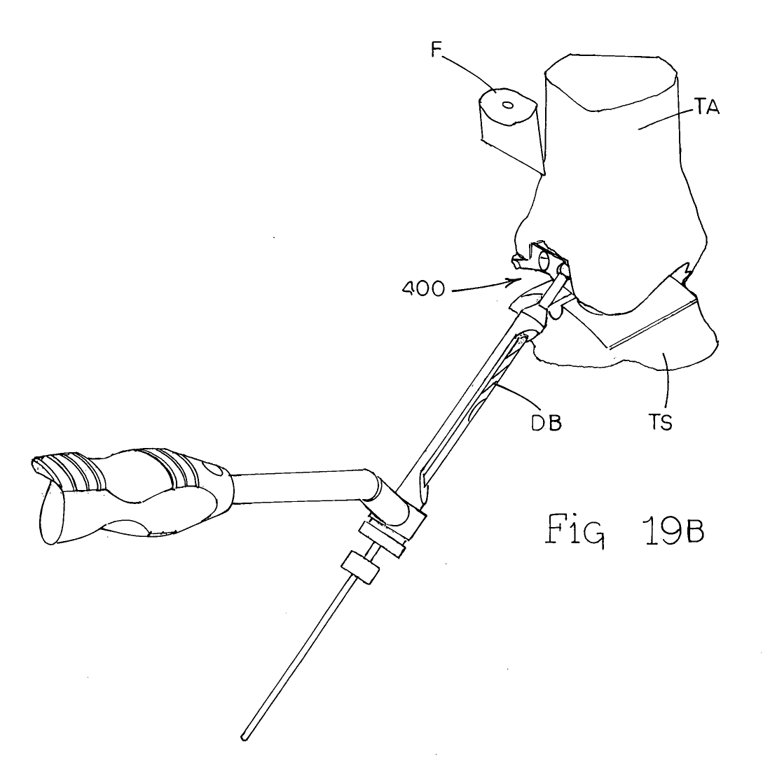

Referring to FIG. 18, the broaching of talus TS using talus broach guide 830 can be made using an appropriate sized drill bit DB, for example a 5.0 mm drill bit, that is passed through recess drill hole 834 of talus broach guide 830 (see also FIG. 8). Drill depth guide DDG can be used to ensure that the proper drill depth is maintained. Drilling through recess drill hole 834 creates a recess on the resected surface of talus bone TA to match a rib profile of preferred talar component 200. Talus broach guide 830 can then be removed from anchor pins AP.

Once the resected surfaces of tibia bone TA and talus bone TS have been prepared, implantation of preferred tibial component 400, talar component 200, and bearing component 300 can occur. Possibly determined by pre-operative anterior/posterior, axial, and medial/lateral scans, appropriate sized tibia, talus and bearing trial components can be placed by the surgeon into the prepared joint to verify the correct implant size and to verify the correct bearing thickness that will be used. Once these sizes are verified, the final implantation can occur.

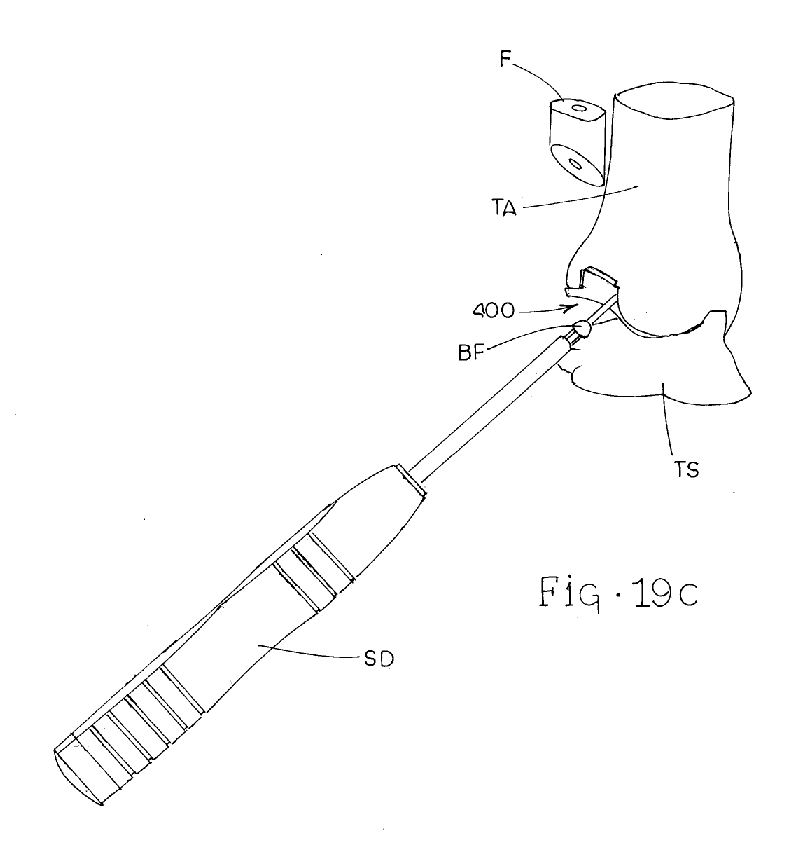

Referring to FIG. 19A, tibial component 400 is inserted into prepared tibia bone TA by using an impactor hand piece I. If necessary, a small mallet (not shown) can be used along with impactor I. Referring to FIG. 19B, using a standard drill guide and drill bit DB, for example a 2.0 mm drill bit, a pilot hole is drilled for locating of appropriate bone fasteners BF. Referring to FIG. 19C, a tibia screw driver SD is used to place the bone fasteners BF into tibia bone TA in order to secure tibial component 400.

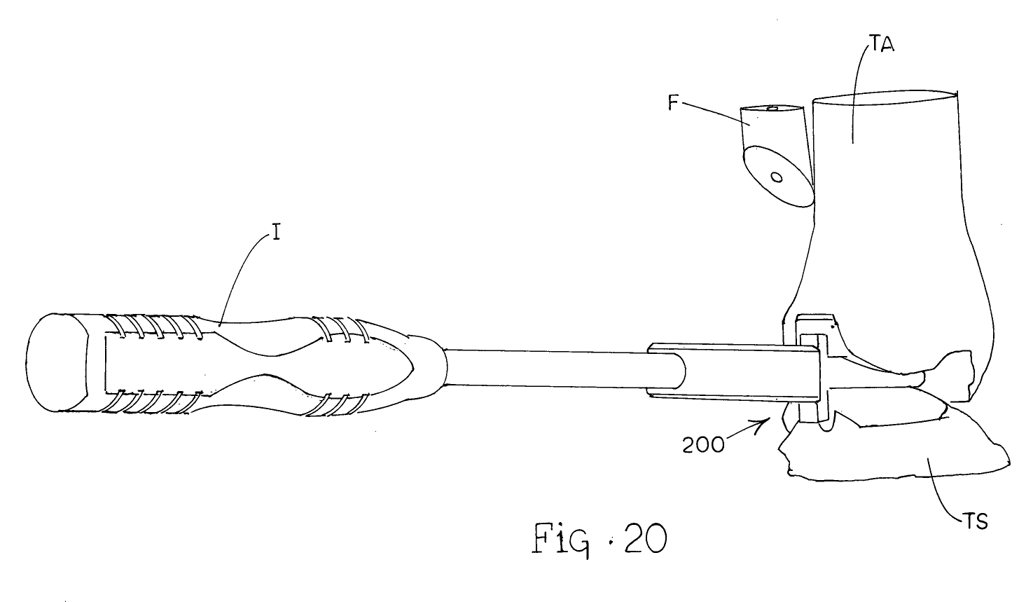

Referring to FIG. 20, talar component 200 can be inserted into prepared talus bone TS by using impactor I. Bearing component 300 can then be placed into sliding engagement between tibial component 400 and talar component 200. Once the installation of the prosthesis components is finished, anchor pins AP can be removed and implantation of the prosthesis is complete.

Once implantation of the prosthesis components is complete, repair of fibula lateral malleolus FM or tibia medial malleolus TM must be performed. If exposure of tibia/talus bone interface TTI and implantation of the prosthesis was performed laterally to medially, current fixation techniques can be performed in order to repair the fibula lateral malleolus FM. Likewise, if exposure of tibia/talus bone interface TTI and implantation of the prosthesis was performed medially to laterally, current fixation techniques can be performed in order to repair tibia medial malleolus TM. Once repair of fibula lateral malleolus FM or tibia medial malleolus TM is completed, current closure techniques can be used to close the incision.

It will be understood that various details of the presently disclosed subject matter may be changed without departing from the scope of the subject matter. Furthermore, the foregoing description is for the purpose of illustration only, and not for the purpose of limitation.

What is claimed is:

1. An ankle prosthesis apparatus comprising:
(a) a talar component for fixation to a talus bone, the talar component having a lower surface and an upper surface;
(b) a tibial component for fixation to a tibia bone and/or a fibula bone, the tibial component having an upper surface with a bone fixation portion, and the tibial component also having a lower surface defining at least one recess;
(c) a bearing component for placement between the tibial and talar components, the bearing component having a lower surface for cooperative engagement with the upper surface of the talar component, and the bearing component also having an upper surface with at least one protrusion; and
(d) wherein the protrusion of the bearing component is adapted to engage the recess of the tibial component to desirably limit rotational and translational movement of the tibial component relative to the bearing component.

2. The ankle prosthesis apparatus of claim 1 wherein the upper surface of the talar component is curved.

3. The ankle prosthesis apparatus of claim 2 wherein the curved upper surface of the talar component is at least generally convex from one side of the talar component to an opposite side of the talar component.

4. The ankle prosthesis apparatus of claim 2 wherein the curved upper surface of the talar component comprises a sulcus arc extending across the upper surface.

5. The ankle prosthesis apparatus of claim 1 wherein the lower surface of the talar component is curved.

6. The ankle prosthesis apparatus of claim 5 wherein the curved lower surface of the talar component is at least generally concave from one side of the talar component to an opposite side of the talar component.

7. The ankle prosthesis apparatus of claim 1 wherein the talar component comprises a rib disposed on the lower surface of the talar component.

8. The ankle prosthesis apparatus of claim 7 wherein the rib extends from one side of the talar component to an opposite side of the talar component.

9. The ankle prosthesis apparatus of claim 8 wherein the talar component extends further from the rib in a posterior direction than in an opposite, anterior direction.

10. The ankle prosthesis apparatus of claim 1 wherein a lateral side height of the talar component is greater than a medial side height of the talar component.

11. The ankle prosthesis apparatus according to claim 1 wherein the lower surface of the bearing component is curved.

12. The ankle prosthesis apparatus according to claim 11 wherein the curved lower surface of the bearing component is at least generally concave from one side of the bearing component to an opposite side of the bearing component.

13. The ankle prosthesis apparatus of claim 11 wherein the lower surface of the bearing component comprises a raised portion extending at least partially across the lower surface of the bearing component.

14. The ankle prosthesis apparatus of claim 1 wherein the upper surface of the bearing component is curved.

15. The ankle prosthesis apparatus of claim 14 wherein the curved upper surface of the bearing component is at least generally convex from one side of the bearing component to an opposite side of the bearing component.

16. The ankle prosthesis apparatus of claim 1 wherein the protrusion on the upper surface of the bearing component comprises a bearing plug extending from the upper surface of the bearing component.

17. The ankle prosthesis apparatus of claim 16 wherein the bearing plug is disposed generally centrally on the upper surface of the bearing component.

18. The ankle prosthesis apparatus of claim 1 wherein a medial side height of the bearing component is greater than a lateral side height of the bearing component.

19. The ankle prosthesis apparatus of claim 1 wherein the upper surface of the tibial component comprises a tibial attachment portion.

20. The ankle prosthesis apparatus of claim 19 wherein the tibial attachment portion comprises a raised shelf.

21. The ankle prosthesis apparatus of claim 19 wherein the tibial attachment portion defines a hole for receiving a bone fastener.

22. The ankle prosthesis apparatus of claim 19 wherein the tibial attachment portion defines a plurality of holes each for receiving a bone fastener.

23. The ankle prosthesis apparatus of claim 19 wherein the tibial attachment portion comprises at least one rib for fixation to a tibial bone and/or a fibula bone.

24. The ankle prosthesis apparatus of claim 1 wherein the recess of the tibial component extends into the tibial attachment portion.

25. The ankle prosthesis apparatus of claim 1 wherein the recess of the tibial component is shaped generally like a rectangle.

26. The ankle prosthesis apparatus of claim 1 wherein the recess of the tibial component is disposed generally centrally on the lower surface of the tibial component.

27. The ankle prosthesis apparatus of claim 1 wherein the protrusion of the bearing component and the recess of the tibial component are shaped to matingly fit together.

28. An ankle prosthesis apparatus comprising:
(a) a talar component for fixation to a talus bone, the talar component having a curved lower surface and a curved upper surface, the lower surface having a bone fixation portion for attachment of the talar component to a talus bone;
(b) a tibial component for fixation to a tibia bone and/or a fibula bone, the tibial component having a curved upper surface and a curved lower surface that defines at least one recess;
(c) a bearing component for placement between the tibial and talar components, the bearing component having a curved lower surface for cooperative engagement with the curved upper surface of the talar component, and the bearing component also having a curved upper surface with at least one protrusion extending from the upper surface; and
(d) wherein the protrusion of the bearing component is adapted to engage the recess of the tibial component to desirably limit rotational and translational movement of the tibial component relative to the bearing component.

29. The ankle prosthesis apparatus according to claim 28 wherein the curved upper surface of the talar component is at least generally convex from one side of the talar component to an opposite side of the talar component.

30. The ankle prosthesis apparatus according to claim 28 wherein the curved upper surface of the talar component above the bone fixation portion comprises a sulcus arc extending across the upper surface in a direction at least generally transverse to a direction in which the bone fixation portion extends.

31. The ankle prosthesis system of claim 28 wherein the curved lower surface of the talar component is at least generally concave from one side of the talar component to an opposite side of the talar component.

32. The ankle prosthesis system of claim 28 wherein the bone fixation portion of the talar component comprises a rib disposed on the lower surface of the talar component and extending from across the lower surface of the talar component.

33. The ankle prosthesis system of claim 32 wherein the talar component extends further from the rib in a posterior direction than in an opposite, anterior direction.

34. The ankle prosthesis system of claim 28 wherein a lateral side height of the talar component is greater than a medial side height of the talar component.

35. The ankle prosthesis apparatus according to claim 25 wherein the curved lower surface of the bearing component is at least generally concave from one side of the bearing component to an opposite side of the bearing component.

36. The ankle prosthesis apparatus of claim 25 wherein the lower surface of the bearing component comprises a raised portion extending across the upper surface of the bearing component.

37. The ankle prosthesis apparatus of claim 25 wherein the curved upper surface of the bearing component is at least generally convex from one side of the bearing component to an opposite side of the bearing component.

38. The ankle prosthesis apparatus of claim 25 wherein the protrusion on the upper surface of the bearing component comprises a bearing plug extending from the upper surface of the bearing component.

39. The ankle prosthesis apparatus of claim 38 wherein the bearing plug is disposed generally centrally on the upper surface of the bearing component.

40. The ankle prosthesis apparatus of claim 28 wherein a medial side height of the bearing component is greater than a lateral side height of the bearing component.

41. The ankle prosthesis apparatus of claim 28 wherein the upper surface of the tibial component comprises a raised shelf portion that defines a hole for receiving a bone fastener.

42. The ankle prosthesis apparatus of claim 41 wherein the raised shelf portion of the tibial component defines a plurality of holes each for receiving a bone fastener.

43. The ankle prosthesis apparatus of claim 28 wherein the upper surface of the tibial component comprises a raised shelf portion that comprises at least one rib for fixation to a tibial bone and/or a fibula bone.

44. The ankle prosthesis apparatus of claim 28 wherein the recess of the tibial component extends into a raised shelf portion that is on the upper surface of the tibial component.

45. The ankle prosthesis apparatus of claim 28 wherein the recess of the tibial component is generally rectangular in shape.

46. The ankle prosthesis apparatus of claim 28 wherein the recess of the tibial component is disposed generally centrally on the lower surface of the tibial component.

47. The ankle prosthesis apparatus of claim 28 wherein the protrusion of the bearing component and the recess of the tibial component are shaped to matingly fit together.

48. An ankle prosthesis apparatus comprising:
(a) a talar component for fixation to a talus bone, the talar component having a curved upper surface and a curved lower surface with a bone fixation portion disposed on the curved lower surface, wherein a first height of the talar component on a lateral side of the talar component extends from a bottom of the bone fixation portion to the upper surface and is greater than a second height of the talar component on a medial side of the talar component that extends from a bottom of the bone fixation portion to the upper surface;
(b) a tibial component for fixation to a tibia bone and/or a fibula bone, the tibial component having an upper surface and a lower surface; and
(c) a bearing component for placement between the tibial and talar components, the bearing component having a lower surface for cooperative engagement with the upper surface of the talar component.

49. The ankle prosthesis apparatus of claim 48 wherein the bone fixation portion comprises a rib that extends from a lateral side of the talar component to an opposite medial side of the talar component.

50. The ankle prosthesis apparatus of claim 49 wherein the upper surface of the talar component comprises a sulcus arc extending across the upper surface and in a direction at least generally transverse to a direction in which the rib extends.

51. A method of implanting an ankle prosthesis apparatus comprising:
(a) providing a talar component having an upper surface and a lower surface with a bone fixation portion for fixation to a talus bone;
(b) providing a bearing component with a lower surface for fitting against the upper surface of the talar component and an upper surface having a protrusion;
(c) providing a tibial component having an upper surface for fixation to a tibia bone and/or a fibula bone, and the tibial component also having a lower surface having at least one recess; and
(d) fitting the protrusion of the bearing component into the recess of the tibial component.

52. A method of implanting an ankle prosthesis apparatus in an ankle joint comprising:
(a) providing a talar component having an upper surface and a lower surface with a bone fixation portion for fixation to a talus bone;
(b) providing a bearing component with a lower surface for fitting against the upper surface of the talar component and an upper surface having a protrusion;
(c) providing a tibial component having an upper surface for fixation to a tibia bone and/or a fibula bone, and the tibial component also having a lower surface having at least one recess; and
(d) implanting the talar component, the bearing component and the tibial component in a medial to lateral approach in an ankle joint.

53. A method of preparing a bone surface for implantation of a prosthesis, the method comprising the steps of:
(a) exposing a bone for implantation;
(b) determining a location and shape for a resection line for resectioning of the bone;
(c) drilling a series of holes tangent to the resection line;
(d) removing a portion of the bone to create a resection surface; and
(e) finishing the resection surface to create a smooth resection surface.

54. The method of claim 53 wherein determining the location and shape for the resection line further comprises determining the location for a curved cut line.

55. The method of claim 54 wherein finishing the resection surface further comprises using a crescentic saw blade.

56. The method of claim 53 wherein drilling the series of holes further comprises drilling a series of holes being adjacent to one another.

57. The method of claim 53 wherein drilling the series of holes further comprises using a drill guide that permits proper positioning of a drill bit for proper alignment of the holes tangent to the resection line.

58. A method of preparing a bone surface for implantation of a prosthesis, the method comprising the steps of:
(a) exposing a bone for implantation;
(b) determining a location for a curved cut line for resection of the bone;
(c) drilling a series of holes tangent to the curved cut line, the holes being adjacent to one another;
(d) removing a portion of the bone to create a curved bone surface; and
(e) using a crescentic saw blade to finish the curved bone surface to create a smooth resection surface.

59. A system for preparing a bone surface for implantation of a prosthesis, the system comprising:
(a) a drill guide defining a plurality of drill holes along a path wherein each drill hole is adapted to receive a drill bit for drilling of holes tangent to a resection line for a bone surface to create a resection surface; and
(b) a saw guide adapted to guide a saw blade for finishing of the resection surface.

60. The system of claim 59 wherein the drill holes of the drill guide are along a curved path.

61. The system of claim 60 wherein the saw guide is adapted to guide a crescentic saw blade.

62. The system of claim 59 wherein the drill holes of the drill guide are adjacent to one another.

63. The system of claim 62 wherein the drill holes are defined as alternating 2.0 millimeter and 3.2 millimeter sized holes.

64. A system for preparing a bone surface for implantation of a prosthesis, the system comprising:
(a) a drill guide defining a plurality of drill holes adjacent to one another along a curved path wherein each drill hole is adapted to receive a drill bit for drilling of holes tangent to a curved cut line for a bone surface to create a curved bone surface; and
(b) a saw guide for guiding a crescentic saw blade for finishing of the curved bone surface.

65. A method of preparing a bone surface for implantation of an ankle joint prosthesis, the method comprising the steps of:
(a) exposing a bone interface for implantation;
(b) determining a location for a resection line for resectioning of a bone selected from the group consisting of a tibia bone, a talus bone, and a fibula bone;
(c) positioning and affixing an alignment guide;
(d) using the alignment guide to provide a template to drill holes tangent to the resection line;
(e) drilling a series of holes tangent to the resection line and removing a portion of the bone to create a resection surface on the bone;
(f) finishing the resection surface of the bone to thereby create a smooth resection surface.

66. The method of claim 65 wherein exposing the bone interface for implantation is performed laterally to medially.

67. The method of claim 65 wherein exposing the bone interface for implantation is performed medially to laterally.

68. The method of claim 65 wherein determining the location for the resection line further comprises determining the location for a curved cut line.

69. The method of claim 68 wherein finishing the resection surface further comprises using a crescentic saw blade.

70. The method of claim 65 wherein drilling the series of holes tangent to the resection line further comprises drilling a series of holes being adjacent to one another.

71. A method of preparing a bone surface for implantation of an ankle joint prosthesis, the method comprising the steps of:
(a) exposing a tibia and talus bone interface for implantation;
(b) determining a location for a tibia curved cut line for resection of the tibia bone and determining a location for a talus curved cut line for resection of the talus bone;
(c) positioning and affixing an alignment guide to the tibia bone;
(d) using the alignment guide to provide a template to drill holes tangent to the tibia and talus cut lines, respectively;
(e) drilling a series of holes tangent to the tibia cut line and removing a portion of the tibia bone to create a curved bone surface on the tibia bone;
(f) drilling a series of holes tangent to the talus cut line and removing a portion of the talus bone to create a curved bone surface on the talus bone; and
(g) finishing the curved bone surfaces of the tibia and talus bones, respectively, to thereby create smooth resection surfaces.

72. The method of claim 71 wherein exposing the tibia and talus bone interface for implantation is performed laterally to medially.

73. The method of claim 72 wherein the lateral to medial exposure further comprises resection of a fibula bone and removal of a portion of the fibula bone whereby the tibia and talus bone interface is exposed.

74. The method of claim 71 wherein exposing the tibia and talus bone interface for implantation is performed medially to laterally.

75. The method of claim 74 wherein the medial to lateral exposure further comprises resection of a medial malleolus portion of the tibia bone and removal of a portion of the medial malleolus whereby the tibia and talus bone interface is exposed.

76. The method of claim 71 wherein drilling the series of holes tangent to the tibia and talus cut lines, respectively, further comprises drilling a series of holes being adjacent to one another.

77. The method of claim 71 wherein finishing the curved bone resection surfaces further comprises using a crescentic saw blade.

78. A method of preparing a bone surface for implantation of an ankle joint prosthesis, the method comprising the steps of:
(a) exposing a tibia and talus bone interface for implantation;
(b) determining a location for a tibia curved cut line for resection of the tibia bone and determining a location for a talus curved cut line for resection of the talus bone;
(c) positioning and affixing an alignment guide to the tibia bone;
(d) using the alignment guide to provide a template to drill holes tangent to the tibia and talus cut lines, respectively;
(e) drilling a series of holes adjacent to one another and tangent to the tibia cut line and removing a portion of the tibia bone to create a curved bone surface on the tibia bone;
(f) drilling a series of holes adjacent to one another and tangent to the talus cut line and removing a portion of the talus bone to create a curved bone surface on the talus bone; and
(g) using a crescentic saw blade to finish the curved bone surfaces of the tibia and talus bones, respectively, to thereby create smooth resection surfaces.

79. A system for preparing a bone surface for implantation of an ankle joint prosthesis, the system comprising:
(a) an alignment guide adapted for alignment with a tibia bone and a talus bone, the alignment guide having at least one fastening hole for receiving a fastener for fastening of the alignment guide to the tibia bone;
(b) a tibia drill guide attached to the alignment guide defining a plurality of drill holes along a curved path wherein each drill hole is adapted to receive a drill bit for drilling of holes tangent to a curved cut line for the tibia bone to create a tibia curved bone surface;
(c) a talus drill guide attached to the alignment guide defining a plurality of drill holes along a curved path wherein each drill hole is adapted to receive a drill bit for drilling of holes tangent to a curved cut line for the talus bone to create a talus curved bone surface;
(d) a tibia saw guide adapted to guide a saw blade for finishing of the tibia curved bone surface; and
(e) a talus saw guide adapted to guide a saw blade for finishing of the talus curved bone surface.

80. The system of claim 79 wherein the tibia and talus drill guides further comprise adjustment knobs adapted for independent adjustment of the tibia and talus drill guides in relation to the alignment guide.

81. The system of claim 79 wherein the drill holes of the tibia and talus drill guides are adjacent to one another.

82. The system of claim 81 wherein the drill holes are defined as alternating 2.0 millimeter and 3.2 millimeter sized holes.

83. The system of claim 79 wherein the tibia and talus saw guides are adapted to guide a crescentic saw blade.

84. The system of claim 79 further comprising an alignment rod attached to the alignment guide and adapted for substantially parallel alignment with a long axis of the tibia bone for alignment of the alignment guide with the tibia bone.

85. The system of claim 84 further comprising an alignment tongue attached to the alignment guide and adapted for insertion between the tibia bone and the talus bone for alignment of the alignment guide with the tibia bone and the talus bone.

86. A system for preparing a bone surface for implantation of an ankle joint prosthesis, the system comprising:
(a) an alignment guide adapted for alignment with a tibia bone and a talus bone, the alignment guide having at least one fastening hole for receiving a fastener for fastening of the alignment guide to the tibia bone;
(b) a tibia drill guide attached to the alignment guide defining a plurality of drill holes adjacent to one another along a curved path wherein each drill hole is adapted to receive a drill bit for drilling of holes tangent to a curved cut line for the tibia bone to create a tibia curved bone surface;
(c) a talus drill guide attached to the alignment guide defining a plurality of drill holes adjacent to one another along a curved path wherein each drill hole is adapted to receive a drill bit for drilling of holes tangent to a curved cut line for the talus bone to create a talus curved bone surface;
(d) a tibia saw guide adapted to guide a crescentic saw blade for finishing of the tibia curved bone surface;
(e) a talus saw guide adapted to guide a crescentic saw blade for finishing of the talus curved bone surface;
(f) an alignment rod attached to the alignment guide and adapted for substantially parallel alignment with a long axis of the tibia bone for alignment of the alignment guide with the tibia bone; and
(g) an alignment tongue attached to the alignment guide and adapted for insertion between the tibia bone and the talus bone for alignment of the alignment guide with the tibia bone and the talus bone.

87. A method of implanting an ankle joint prosthesis comprising tibial, talar and bearing components between distal tibia and talus bones, the method comprising the steps of:
(a) exposing a tibia bone and a talus bone interface for implantation;
(b) determining a location for a tibia curved cut line for resection of the tibia bone and determining a location for a talus curved cut line for resection of the talus bone;
(c) positioning and affixing an alignment guide to the tibia bone;
(d) positioning and affixing a plurality of alignment anchors to the tibia and talus bones;
(e) using the alignment guide to provide a template to drill holes tangent to the tibia and talus cut lines, respectively;
(f) drilling a series of holes tangent to the tibia cut line and removing a portion of the tibia bone to create a curved bone resection surface on the tibia bone;
(g) drilling a series of holes tangent to the talus cut line and removing a portion of the talus bone to create a curved bone resection surface on the talus bone;
(h) removing the alignment guide while leaving the alignment anchors in place;
(i) positioning additional guide components on the alignment anchors for guiding a saw blade for finishing of the tibia and talus resection surfaces, guiding instruments for broaching of the tibia bone to form a tibia broach, and guiding instruments for broaching of the talus bone to form a talus broach;
(j) removing the alignment anchors from the tibia and talus bones;
(k) positioning and affixing a tibial component within the tibia broach so that a top surface of the tibial component abuts and is adjacent to the resected tibia bone;
(l) positioning and affixing a talar component within the talus broach so that a bottom surface of the talar component abuts and is adjacent to the resected talus bone; and
(m) placing a bearing component between the tibial component and the talar component to desirably provide rotational and translational movement of the tibial component relative to the talar component, wherein a top surface of the bearing component slidably engages a bottom surface of the tibial component and a bottom surface of the bearing component slidably engages a top surface of the talar component.

88. The method of claim 87 wherein exposing the tibia and talus bone interface for implantation is performed laterally to medially.

89. The method of claim 88 wherein the lateral to medial exposure further comprises resection of a fibula bone and removal of a portion of the fibula bone whereby the tibia and talus bone interface is exposed.

90. The method of claim 87 wherein exposing the tibia and talus bone interface for implantation is performed medially to laterally.

91. The method of claim 90 wherein the medial to lateral exposure further comprises resection of a medial malleolus portion of the tibia bone and removal of a portion of the medial malleolus whereby the tibia and talus bone interface is exposed.

92. The method of claim 87 wherein positioning and affixing of the plurality of alignment anchors comprises positioning and affixing two alignment anchors to the tibia bone and two alignment anchors to the talus bone.

93. The method of claim 87 wherein drilling the series of holes tangent to the tibia and talus cut lines, respectively, further comprises drilling a series of holes being adjacent to one another.

94. The method of claim 87 wherein finishing of the tibia and talus resection surfaces further comprises using a crescentic saw blade.

95. The method of claim 87 wherein broaching of the tibia bone further comprises cutting a recess that matches the profile of the top surface of the tibial component.

96. The method of claim 87 wherein broaching of the talus bone further comprises cutting a recess that matches the profile of the bottom surface of the talar component.

97. The method of claim 87 further comprising the step of taking pre-operative images of the ankle area to determine a size of the prosthesis to be used.

98. A method of implanting an ankle joint prosthesis comprising tibial, talar and bearing components between distal tibia and talus bones, the method comprising the steps of:
(a) exposing a tibia bone and a talus bone interface for implantation;
(b) determining a location for a tibia curved cut line for resection of the tibia bone and determining a location for a talus curved cut line for resection of the talus bone;
(c) positioning and affixing an alignment guide to the tibia bone;
(d) positioning and affixing a plurality of alignment anchors to the tibia and talus bones;
(e) using the alignment guide to provide a template to drill holes tangent to the tibia and talus cut lines, respectively;
(f) drilling a series of holes adjacent to one another and tangent to the tibia cut line and removing a portion of the tibia bone to create a curved bone resection surface on the tibia bone;
(g) drilling a series of holes adjacent to one another and tangent to the talus cut line and removing a portion of the talus bone to create a curved bone resection surface on the talus bone;
(h) removing the alignment guide while leaving the alignment anchors in place;
(i) positioning additional guide components on the alignment anchors for guiding a crescentic saw blade for finishing of the tibia and talus resection surfaces, guiding instruments for broaching of the tibia bone to form a tibia broach, and guiding instruments for broaching of the talus bone to form a talus broach;
(j) removing the alignment anchors from the tibia and talus bones;
(k) positioning and affixing a tibial component within the tibia broach so that a top surface of the tibial component abuts and is adjacent to the resected tibia bone;
(l) positioning and affixing a talar component within the talus broach so that a bottom surface of the talar component abuts and is adjacent to the resected talus bone; and
(m) placing a bearing component between the tibial component and the talar component to desirably provide rotational and translational movement of the tibial component relative to the talar component, wherein a top surface of the bearing component slidably engages a bottom surface of the tibial component and a bottom surface of the bearing component slidably engages a top surface of the talar component.

99. The method of claim 98 wherein exposing the tibia and talus bone interface for implantation is performed laterally to medially.

100. The method of claim 98 wherein exposing the tibia and talus bone interface for implantation is performed medially to laterally.

101. A system for implanting an ankle joint prosthesis comprising tibial, talar and bearing components between distal tibia and talus bones, the system comprising:
(a) an alignment guide adapted for alignment with a tibia bone and a talus bone, the alignment guide having at least one fastening hole for receiving a fastener for fastening of the alignment guide to the tibia bone;
(b) a tibia drill guide attached to the alignment guide defining a plurality of drill holes along a curved path wherein each drill hole is adapted to receive a drill bit for drilling of holes tangent to a curved cut line on the tibia bone to create a tibia curved bone surface;
(c) a talus drill guide attached to the alignment guide defining a plurality of drill holes along a curved path wherein each drill hole is adapted to receive a drill bit for drilling of holes tangent to a curved cut line on the talus bone to create a talus curved bone surface;
(d) a tibia saw guide for guiding a saw blade for finishing of the tibia curved bone surface;
(e) a talus saw guide for guiding a saw blade for finishing of the talus curved bone surface;
(f) a tibia broach guide for guiding instruments for broaching of the tibia bone;
(g) a talus broach guide for guiding instruments for broaching of the talus bone; and
(h) a plurality of alignment anchors for guiding the tibia and talus drill guides, tibia and talus saw guides, and tibia and talus broach guides.

102. The system of claim 101 wherein the tibia and talus drill guides further comprise adjustment knobs adapted for independent adjustment of the tibia and talus drill guides in relation to the alignment guide.

103. The system of claim 101 wherein the drill holes of the tibia and talus drill guides are adjacent to one another.

104. The system of claim 103 wherein the drill holes are defined as alternating 2.0 millimeter and 3.2 millimeter sized holes.

105. The system of claim 101 wherein the tibia and talus saw guides are for guiding a crescentic saw blade.

106. The system of claim 101 wherein the tibia broach guide comprises a recess, wherein the tibia broach guide is adapted to guide instruments for broaching of the tibia bone including cutting a recess that matches a top surface profile of a tibial component.

107. The system of claim 101 wherein the talus broach guide comprises a recess, wherein the talus broach guide is adapted to guide instruments for broaching of the talus bone including cutting a recess that matches a bottom surface profile of a talar component.

108. The system of claim 101 further comprising an alignment rod attached to the alignment guide and adapted for substantially parallel alignment with a long axis of the tibia bone for alignment of the alignment guide with the tibia bone.

109. The system of claim 108 further comprising an alignment tongue attached to the alignment guide and adapted for insertion between the tibia bone and the talus bone for alignment of the alignment guide with the tibia bone and the talus bone.

110. A system for implanting an ankle joint prosthesis comprising tibial, talar and bearing components between distal tibia and talus bones, the system comprising:
(a) an alignment guide adapted for alignment with a tibia bone and a talus bone, the alignment guide having at least one fastening hole for receiving a fastener for fastening of the alignment guide to the tibia bone;
(b) a tibia drill guide attached to the alignment guide defining a plurality of drill holes adjacent to one another along a curved path wherein each drill hole is adapted to receive a drill bit for drilling of holes tangent to a curved cut line on the tibia bone to create a tibia curved bone surface;
(c) a talus drill guide attached to the alignment guide defining a plurality of drill holes adjacent to one another along a curved path wherein each drill hole is adapted to receive a drill bit for drilling of holes tangent to a curved cut line on the talus bone to create a talus curved bone surface;
(d) a tibia saw guide for guiding a crescentic saw blade for finishing of the tibia curved bone surface;
(e) a talus saw guide for guiding a crescentic saw blade for finishing of the talus curved bone surface;
(f) a tibia broach guide comprising a recess, wherein the tibia broach guide is adapted to guide instruments for broaching of the tibia bone including cutting a recess that matches a top surface profile of a tibial component;
(g) a talus broach guide comprising a recess, wherein the talus broach guide is adapted to guide instruments for broaching of the talus bone including cutting a recess that matches a bottom surface profile of a talar component;
(h) a plurality of alignment anchors for guiding the tibia and talus drill guides, tibia and talus saw guides, and tibia and talus broach guides;
(i) an alignment rod attached to the alignment guide for substantially parallel alignment with a long axis of the tibia bone for alignment of the alignment guide with the tibia bone; and
(j) an alignment tongue attached to the alignment guide for insertion between the tibia bone and the talus bone for alignment of the alignment guide with the tibia bone and the talus bone.

111. A method for medially to laterally implanting an ankle joint prosthesis comprising tibial, talar and bearing components between distal tibia and talus bones, the method comprising the steps of:
(a) surgically opening a medial side of an ankle area and exposing a tibia bone;
(b) resection of a medial malleolus located on the distal end of the tibia bone, the resection being along a plane that is generally parallel with the longitudinal axis of the tibia bone and the resection exposing a talus bone;
(c) resection of the tibia bone;
(d) resection of the talus bone;
(e) broaching the tibia bone to form a tibia broach;
(f) broaching the talus bone to form a talus broach;
(g) positioning and affixing a tibial component within the tibia broach so that a top surface of the tibial component abuts and is adjacent to the resected tibia bone;
(h) positioning and affixing a talar component within the talus broach so that a bottom surface of the talar component abuts and is adjacent to the resected talus bone;
(i) placing a bearing component between the tibial component and the talar component to desirably provide rotational and translational movement of the tibial component relative to the talar component, wherein a top surface of the bearing component slidably engages a bottom surface of the tibial component and a bottom surface of the bearing component slidably engages a top surface of the talar component;
(j) replacing the resected medial malleolus portion; and
(k) closing the ankle area.

112. The method of claim 111 wherein resection and broaching of the tibia bone and the talus bone further comprise using cutting guides that permit proper positioning of the required cutting implements.

113. The method of claim 111 further comprising the step of taking pre-operative images of the ankle area to determine a size of the prosthesis to be used.
